(19) United States
(12) Patent Application Publication
(10) Pub. No.: US 2026/0191420 A1
(43) Pub. Date: Jul. 9, 2026

(54) SYSTEM AND METHOD FOR TREATMENT OF MEDICAL CONDITIONS USING MULTIPLE BLOOD PRESSURE SENSORS

(71) Applicant: Aktiia SA, Neuchâtel (CH)

(72) Inventors: Josep Sola, Cormondrèche (CH); Jay Shah, Arlington, MA (US); Calvin Limat, Düdingen (CH); Tiago Paggi De Almeida, Zug (CH); David Perruchoud, Uster (CH); Grégoire Wuerzner, Le Mont-Sur-Lausanne (CH); Olivier Grossenbacher, Neuchâtel (CH); Raghav Gupta, Winchester, MA (US)

(21) Appl. No.: 19/294,048

(22) Filed: Aug. 7, 2025

Related U.S. Application Data
(62) Division of application No. 19/012,213, filed on Jan. 7, 2025.

(51) Int. Cl. A61B 5/021; A61B 5/00; A61B 5/02; G16H 20/00

(52) U.S. Cl. CPC A61B 5/021; A61B 5/02028; A61B 5/6803; A61B 5/681; A61B 5/6891; A61B 5/6893; A61B 5/6898; A61B 5/742; G16H 20/00; A61B 2560/0462

(57) ABSTRACT

A system for monitoring blood pressure (BP) of a user including a first sensor adapted to measure BP of the user and screen users for candidates to use a second sensor. The second sensor is: (i) of a different type than the first sensor, and (ii) adapted to measure BP of the user periodically and without user initiation.

### TECHNICAL FIELD

This specification relates to systems and methods for treating medical conditions using multiple blood pressure sensors, and in particular, to the treatment of medical conditions using non-invasive blood pressure sensors.

### BACKGROUND

Hypertension (HTN) is the foremost preventable cause of death worldwide, serving as a direct and potent contributor to cardiovascular, neurological, and renal diseases, as well as other serious health conditions. Early detection and personalized management are critical in mitigating these health risks and reducing associated healthcare costs. However, traditional methods for diagnosing and monitoring blood pressure (BP) rely on sporadic measurements of systolic and diastolic blood pressure (SBP and DBP). SBP measures the force in the arteries during heart beats, while DBP measures force in the arteries between heart beats. Such measurements are typically collected using a device called a sphygmomanometer and recorded in millimeters of mercury (mmHg). Traditionally, an inflatable cuff is placed around the upper arm, inflated to restrict blood flow, and a stethoscope is used to listen as air is released to determine the SBP and DBP readings. This process has been automated with digital devices to perform a similar measurement as a human listening with a stethoscope. Due to the need for such specialized equipment, most individuals have their blood pressure measured on a relatively infrequent basis (e.g., when visiting a doctor). As such, the traditional approach results in infrequent and intermittent readings that often fail to accurately capture an individual's physiological BP patterns over time, leading to poor awareness, delayed or missed diagnoses, and suboptimal management of HTN and its related conditions.

### SUMMARY

At least one aspect of the present disclosure is directed to a system for monitoring blood pressure (BP) of a user. The system includes a first sensor adapted to measure BP of the user and screen users for candidates to use a second sensor. The second sensor is: (i) of a different type than the first sensor and (ii) adapted to measure BP of the user periodically and without user initiation.

In some embodiments, the first sensor is adapted to perform a spot check BP measurement. In some embodiments, the first sensor is selected from a group of: a camera, a wearable device, a kiosk, and a toilet sensor. In some embodiments, the first sensor comprises a smartphone camera. In some embodiments, the second sensor is adapted to perform continuous or semi-continuous BP measurements. In some embodiments, the second sensor is further adapted to classify users according to risk profiles. In some embodiments, the second sensor is further adapted to provide personalized recommendations to the user for managing a BP condition. In some embodiments, the second sensor is selected from a group of: a wearable device, a bed sensor, a camera, and a steering wheel sensor. In some embodiments, the second sensor comprises a wearable bracelet. In some embodiments, the first sensor measures BP by measuring only mmHg and wherein the second sensor measures BP using a metric selected from the group consisting of time-in-target range (TTR) and cumulative blood pressure load (CBPL). In some embodiments, the first sensor is further adapted to facilitate the user ordering the second sensor.

Another aspect of the present disclosure is directed to a method for monitoring blood pressure (BP) of a user. The method includes receiving multiple BP measurements for the user during a time period and using the multiple BP measurements to determine at least one of (i) a time-in-target range (TTR) and (ii) a cumulative blood pressure load (CPBL).

In some embodiments, the multiple BP measurements are received from a wearable sensor. In some embodiments, the multiple BP measurements comprise continuous or semi-continuous measurements. In some embodiments, the time period is selected from the group consisting of a day, a week, a month, a year, and multiple years. In some embodiments, the time period corresponds to (i) a duration of a planned intervention related to the user's BP or (ii) a duration of a phase of a planned intervention related to the user's BP. In some embodiments, the method includes presenting the TTR and/or CBPL value to the user. In some embodiments, the method includes providing the user with a recommended action based on the determined TTR and/or CBPL value.

Another aspect of the present disclosure is directed to a method for managing a health condition of a user. The method includes receiving physiological information related to the health condition of the user, presenting the physiological information to the user, tracking data related to user engagement with the physiological information, and providing the user with a recommended action for management of the health condition based on the tracked user engagement data.

In some embodiments, the health condition includes a BP condition. In some embodiments, the physiological information includes a BP measurement. In some embodiments, the BP measurement includes at least one of TTR and CBPL. In some embodiments, presenting the physiological information to the user includes presenting the information on a graphical user interface. In some embodiments, the user engagement data includes a time when the user engages with the physiological data. In some embodiments, the time includes at least one of a time of day, a day of the week, a week of the month, and a day of the year. In some embodiments, the user engagement data further includes at least one of frequency and length of engagement for when the user engages with the physiological information. In some embodiments, the user engagement data further includes an activity that the user performs when the user is engaging with the physiological information. In some embodiments, providing the user with the recommended action for management of the health condition based on the tracked user engagement data includes determining the recommended action based on an engagement pattern of the user. In some embodiments, the engagement pattern of the user is determined based on the received physiological information related to the health condition of the user. In some embodiments, the engagement pattern of the user is determined based on an engagement pattern or physiological information of at least one other user.

### BRIEF DESCRIPTION OF THE DRAWINGS

The accompanying figures, which are included as part of the present specification, illustrate the presently preferred embodiments and together with the general description given above and the detailed description of the preferred embodiments given below serve to explain and teach the principles described herein.

FIG. 1 is a block diagram of a blood pressure monitoring system in accordance with aspects described herein;

FIG. 2 is a block diagram of a portion of the blood pressure monitoring system of FIG. 1 in accordance with aspects described herein;

FIG. 3 is a graph of a blood pressure signal in accordance with aspects described herein;

FIG. 4 is a flow diagram of a method for utilizing advanced blood pressure metrics in accordance with aspects described herein;

FIG. 5 is a flow diagram of a method for calculating an advanced blood pressure metric in accordance with aspects described herein;

FIG. 6 is a flow diagram of a method for calculating an advanced blood pressure metric in accordance with aspects described herein;

FIG. 7 is a flow diagram of a method for calculating an advanced blood pressure metric in accordance with aspects described herein;

FIG. 8 is a flow diagram of a method for calculating an advanced blood pressure metric in accordance with aspects described herein;

FIG. 9 is a flow diagram of a method for calculating an advanced blood pressure metric in accordance with aspects described herein;

FIG. 10 is a flow diagram of a method for calculating an advanced blood pressure metric in accordance with aspects described herein;

FIG. 11 is a flow diagram of a method for calculating an advanced blood pressure metric in accordance with aspects described herein;

FIG. 12 is a flow diagram of a method for calculating an advanced blood pressure metric in accordance with aspects described herein;

FIG. 13 is a flow diagram of a method for calculating an advanced blood pressure metric in accordance with aspects described herein;

FIG. 14 is a flow diagram of a method for calculating an advanced blood pressure metric in accordance with aspects described herein;

FIG. 15 is a flow diagram of a method for calculating an advanced blood pressure metric in accordance with aspects described herein;

FIG. 16 is a flow diagram of a method for calculating an advanced blood pressure metric in accordance with aspects described herein;

FIG. 17 is a flow diagram of a method for calculating an advanced blood pressure metric in accordance with aspects described herein;

FIG. 18 is a flow diagram of a method for calculating an advanced blood pressure metric in accordance with aspects described herein;

FIG. 19 is a flow diagram of a method for calculating an advanced blood pressure metric in accordance with aspects described herein;

FIG. 20 is a flow diagram of a method for calculating an advanced blood pressure metric in accordance with aspects described herein;

FIG. 21 is a flow diagram of a method for generating a visual representation of an advanced blood pressure metric in accordance with aspects described herein;

FIG. 22 is an example plot of advanced blood pressure metrics in accordance with aspects described herein;

FIG. 23 is a flow diagram of a method for calculating an advanced blood pressure metric in accordance with aspects described herein;

FIG. 24 is a flow diagram of a method for calculating an advanced blood pressure metric in accordance with aspects described herein;

FIG. 25 is a flow diagram of a method for calculating an advanced blood pressure metric in accordance with aspects described herein;

FIG. 26 is a flow diagram of a method for calculating an advanced blood pressure metric in accordance with aspects described herein;

FIG. 27 is a flow diagram of a method for calculating an advanced blood pressure metric in accordance with aspects described herein;

FIG. 28 is a flow diagram of a method for calculating an advanced blood pressure metric in accordance with aspects described herein;

FIG. 29 is a flow diagram of a method for generating a visual representation of an advanced blood pressure metric in accordance with aspects described herein;

FIG. 30 is a flow diagram of a method for calculating an advanced blood pressure metric in accordance with aspects described herein;

FIG. 31 is a flow diagram of a method for tracking user engagement with physiological information of a user in accordance with aspects described herein;

FIG. 32A is a diagram including example plots of blood pressure values and engagement patterns in accordance with aspects described herein;

FIG. 32B is a diagram including an example plot of engagement patterns in accordance with aspects described herein; and

FIG. 33 illustrates an example computing device.

While the present disclosure is subject to various modifications and alternative forms, specific embodiments thereof have been shown by way of example in the drawings and will herein be described in detail. The present disclosure should not be understood to be limited to the particular forms disclosed, but on the contrary, the intention is to cover all modifications, equivalents, and alternatives falling within the spirit and scope of the present disclosure.

### DETAILED DESCRIPTION

As described above, the traditional approach to BP management results in infrequent and intermittent readings that often fail to accurately capture an individual's physiological BP patterns over time, leading to poor awareness, delayed or missed diagnoses, and suboptimal management of HTN and its related conditions. The embodiments of this disclosure address these challenges by introducing a comprehensive therapeutic system designed to enhance screening and improve the speed, accuracy, and efficacy of HTN diagnoses, as well as to optimize subsequent management strategies through personalized interventions. The embodiments of this disclosure provide for a system that calculates and presents to users and physicians, in real-time, BP metrics that cannot be produced by traditional BP measurement instruments and techniques. In some embodiments, the system integrates widely accessible devices with advanced monitoring technologies and sophisticated algorithms to provide a seamless and effective approach to BP management.

In some embodiments, the system employs a two-tiered independent approach to monitor and manage a user's BP effectively. A first sensor (e.g., a smartphone camera) performs spot-check BP measurements. When the user places their finger over the camera lens, an application on the smartphone detects pulsatility components of the blood to estimate BP. This method provides an accessible and convenient way for users to perform initial screenings. The application not only measures BP but also classifies and qualifies users for the next level of monitoring based on their readings.

In some embodiments, upon identifying users who may benefit from advanced monitoring, the system introduces a second sensor (e.g., a wearable device). The wearable device may be a wrist-worn bracelet that performs approximately 30 BP readings every 24 hours without user initiation, providing continuous or semi-continuous monitoring. The wearable leverages photoplethysmography (PPG) technology to collect accurate BP measurements throughout the day and night, capturing fluctuations that spot-check measurements might miss. Any other form factor might also be possible such as rings, ear devices or any other wearable device. The second sensor integrates sophisticated algorithms to analyze the collected BP data, giving both traditional BP measurements as well as focusing on advanced therapeutic markers such as the Time-in-Target Range (TTR) and Hi-Load (or cumulative BP load). These metrics offer a comprehensive view of the user's BP diagnosis, responses to interventions, and control over time and correlate with organ-specific risks for conditions like heart disease, stroke, and kidney damage.

In some embodiments, to maximize user engagement, compliance with, and adherence to long-term BP management, the system tracks data related to user interaction with their physiological information. It identifies user-specific “attention windows”-periods when the user is most receptive to health-related communications based on their engagement patterns with the application. Utilizing these insights, the system tailors engaging messages, prompts interactive screens, and delivers personalized content during these optimal times. The user experience is further enhanced through gamified dynamic interventions targeting the optimization of the Hi-Load parameter. The application presents challenges, rewards, and progress tracking features that motivate the user to actively participate in managing their BP. By visualizing the current personal cardiovascular risk derived from the Hi-Load and the potential reduction in personal cardiovascular risk achieved through adherence to recommended actions, the system empowers users to make informed decisions about their health behaviors. For instance, the app may display how consistent use of the wearable and adherence to lifestyle recommendations decreased their Hi-Load value, thereby lowering the risk of a cardiovascular event. This personalized feedback loop reinforces positive behaviors and encourages sustained engagement with the monitoring system.

By utilizing measurements from the first and second sensor, the system provides a comprehensive solution for early detection, accurate diagnosis, and effective management of hypertension, ultimately aiming to improve health outcomes and reduce the global burden of BP-related diseases.

### Monitoring System

A BP monitoring system is provided herein that includes at least two types of sensors. The first sensor performs spot-check BP measurements using accessible devices, such as smartphones, cameras, wearable devices, smart rings, wearable smart bands, kiosks, or toilet sensors. The first sensor may be referred to as “Means A.” The first sensor is widely distributable and facilitates widespread screening and early detection of abnormal BP measurements across large populations. The second sensor performs repeated, consistent, passive, and automated BP measurements without user initiation utilizing devices such as wearable bracelets, bands, rings, bed sensors, integrated steering wheel sensors, or the like. The second sensor may be referred to as “Means B.” In some examples, the second sensor is of a different type than the first sensor; however, in other examples, the second sensor may be the same type of sensor as the first sensor. In some examples, the second sensor corresponds to a different configuration of the first sensor. The automated monitoring performed by the second senor captures continuous or semi-continuous BP data, enabling a more accurate assessment of an individual's BP over time and supports the calculation of advanced metrics.

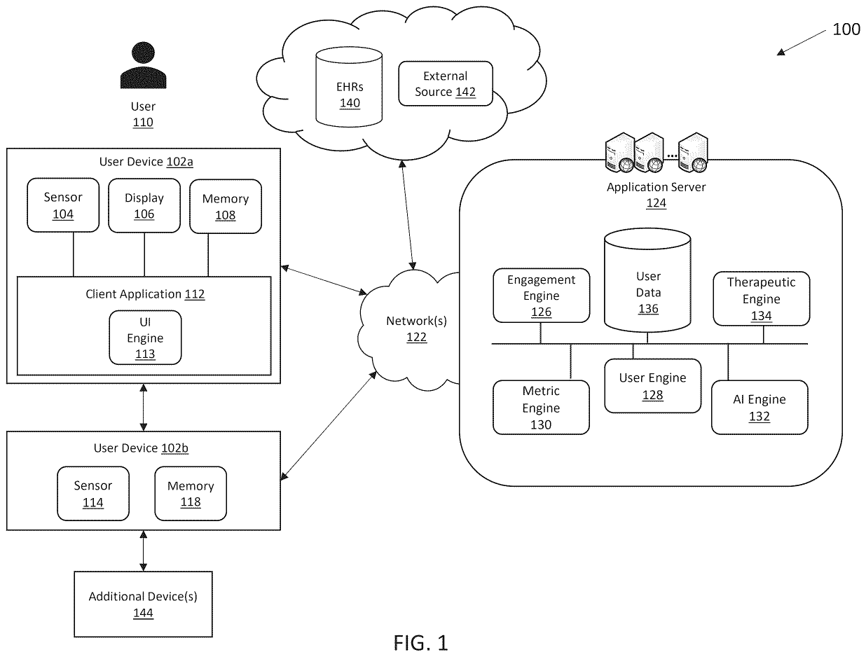

FIG. 1 is a block diagram of an example blood pressure monitoring system 100 in accordance with aspects described herein. The system 100 includes a first user device 102a, a second user device 102b, and an application server 124. In some examples, the first user device 102a is a widely accessible device intended for large-scale deployment to screen extensive populations for potential candidates requiring further monitoring. The first user device 102a performs spot-check BP measurements and is designed for ease of use and widespread availability. In some examples, the first user device 102a performs spot-check measurements other than BP measurements, including a cardiovascular risk measurement, a hypertension risk measurement, a hypotension risk measurement, any measurement of health risk, health condition, or physiological information, or any other measurement that can be used to screen users. The key characteristic is its capacity to be exposed to large populations, enabling early detection through accessible means across broad communities. The first user device 102a may be a device personally owned by the user 110, a public device, or a device owned by a third party (e.g., a medical provider or practitioner). In some examples, the first user device 102a is a smartphone, a computer, a tablet, a smartwatch, a camera, a wearable device, a fitness tracker, a smart ring, a wearable smart band, a kiosk, a toilet sensor, a wearable smart device, a non-wearable smart device, a smart device embedded within another item or device, or a dedicated medical device. Other types of user devices are possible.

In some examples, the first user device 102a includes at least one first sensor 104, at least one display 106, and at least one memory element 108. The first user device 102a may have a housing that includes the first sensor 104. The first sensor 104 corresponds to the “Means A” sensor. In some examples, the first sensor 104 is configured to receive (or collect) a pulsatility signal from a user 110. The pulsatility signal may be a PPG signal from a photoplethysmographic sensor, or any other pulsatility sensor or array of sensors, such as, but not limited to, a bioimpedance sensor, an ultra-sound sensor, a magnetic sensor, a radar sensor, a sensor based on radio frequency, a mechanical sensor, a volume sensor, a non-invasive sensor, an invasive sensor, an intra-arterial sensor, a minimal invasive sensor, a subcutaneous sensor, a tonometer sensor, a strain sensor, a plethysmographic sensor, a microphone, a capacitive sensor, an electromagnetic sensor, a Raman sensor, or any sensor capable of measuring a pulsatility signal either from a capillary bed of the skin or from any other section of the arterial tree. In some examples, the pulsatility signal is a unidimensional pulsatility signal. In some examples, the signal is derived from an image, a series of images, or a video captured by the first sensor 104.

It should be appreciated that the first user device 102a may be any device that is suitable for screening large populations of users, such as security cameras, thermal imaging cameras, facial recognition kiosks, airport body scanners, interactive screens in public spaces, smart mirrors in public restrooms or stores, motion detectors in public spaces, automated teller machine (ATM) kiosks (and cameras), traffic light radars, drones with remote sensors, and sensors used for border control purposes. Likewise, the first user device 102a (or the first sensor 104) may be, or integrated within, any device, item, or object that users interact with or otherwise encounter in everyday life. Examples of such include public transportation seats or handrails, cinema or theater chairs, hairdresser or barber chairs, office chairs, shopping carts, exercise equipment (e.g., treadmills, bikes, etc.), public benches, restaurant chairs, gaming chairs, elevator or escalator handrails, supermarket checkout counters, taxi or rideshare seats, airport seating, stadium seating, interactive kiosks, recliners in lounges, turnstiles, waiting room chairs, library chairs, car steering wheels, smart streetlights with embedded sensors, smart city lamp posts, digital signage displays, vehicle cameras and sensors, or any other suitable device, item, or object.

Examples of techniques for measuring BP using the first user device 102a (or the first sensor 104) are described in PCT Application No. PCT/EP2024/081921, filed on Nov. 11, 2024 and titled “SYSTEM AND METHOD FOR CALIBRATIONLESS DETECTION OF BLOOD PRESSURE WITH VIDEO ACQUISITION PLATFORM,” the entirety of which is incorporated herein by reference.

In some examples, a client application 112 is configured to run (or operate) on the first user device 102a. The client application 112 may be configured to run in a web browser or a special-purpose software application executing on the first user device 102a (e.g., a smartphone application). In some examples, the client application 112 a user interface (UI) engine 113. The client application 112 is configured to process, at least in part, the signal received by the sensor 104 to determine blood pressure values of the user 110. The first user device 102a (or the client application 112) is configured to communicate with the application server 124 through one or more data communication networks 122 such as the Internet, for example. The client application 112 may communicate with the application server 124 over the network 122 using Hypertext Transfer Protocol (HTTP), another standard protocol, or a proprietary protocol, for example. The application server 124 provides functionality for processing data (e.g., pulsatility signals) and storing data (e.g., user data, signal data, etc.).

The application server 124 comprises software components and databases that can be deployed at one or more data centers (not shown) in one or more geographic locations, for example. The application server 124 software components include an engagement engine 126, a user engine 128, a metric engine 130, an artificial intelligence (AI) engine 132, and a therapeutic engine 134. The software components can comprise subcomponents that can execute on the same or on a different individual data processing apparatus. The application server 124 databases include a user data database 136. The databases can reside in one or more physical storage systems. Example features of the software components and data processing apparatus will be further described below. It should be appreciated that the application server 124 may include or otherwise support additional engines and tools. For example, the application server 102 may support third-party tools that provide additional functions to the engines 126-134 and/or the database 136. In some examples, the application server 124 is configured to utilize one or more Application Programming Interfaces (APIs) to communicate with third-party tools and engines.

Although this disclosure will describe many functions as being performed by client application 112, in various implementations, some or all functions performed by the client application 112 may be performed remotely by the application server 124. Likewise, in various implementations, some or all functions performed by the application server 124 may be performed locally by the client application 112.

In some examples, the second user device 102b differs from the first user device 102a in both type and functionality. For example, the second user device 102b may be a wearable bracelet, a band, a ring, a smartwatch, glasses, a bed sensor, or an integrated steering wheel sensor. Other types of user devices are possible. In some examples, the second user device 102b includes at least one second sensor 114 and at least one memory element 118. While not shown, the second user device 102b may include a display. The second user device 102b is configured to provide repeated, passive, and automated BP measurements without requiring any user initiation, delivering continuous or semi-continuous BP data while the second sensor 114 is in contact with the user 110.

It should be appreciated that the second user device 102b (or the second sensor 114) may be, or integrated within, any device, item, or object that is in continuous or close contact with the user's body. Examples of such include clothing, sensors embedded in clothing, sensors embedded on the user's house, mirrors, shoes, hats, glasses, smartphones, smartphone accessories, personal computers, webcams, cameras, computer accessories, tablets, cases, sockets, socks, hearing aids, jewelry, headphones, earbuds, gaming headsets, virtual reality headsets, smart glasses, bracelets, or any other suitable device, item, or object.

The second sensor 114 corresponds to the “Means B” sensor. In some examples, the second sensor 114 is a different type of sensor than the first sensor 104; however, in other examples, the first and second sensors 104, 114 may be the same type of sensor. In some examples, the second sensor 114 is configured to receive (or collect) a pulsatility signal from the user 110. The pulsatility signal may be a PPG signal from a photoplethysmographic sensor, or any other pulsatility sensor or array of sensors, such as, but not limited to, a bioimpedance sensor, an ultra-sound sensor, a magnetic sensor, a radar sensor, a sensor based on radio frequency, a mechanical sensor, a volume sensor, a non-invasive sensor, an invasive sensor, an intra-arterial sensor, a minimal invasive sensor, a subcutaneous sensor, a tonometer sensor, a strain sensor, a plethysmographic sensor, a microphone, a capacitive sensor, an electromagnetic sensor, a Raman sensor, or any sensor capable of measuring a pulsatility signal either from a capillary bed of the skin or from any other section of the arterial tree. In some examples, the pulsatility signal is a unidimensional pulsatility signal. In some examples, the signal is derived from an image, a series of images, or a video captured by the second sensor 114.

In some examples, the second sensor 114 (or the second user device 102b) is configured to communicate with the client application 112 of the first user device 112 (e.g., over a wireless connection, Bluetooth, etc.). In such examples, the client application 112 may process, at least in part, signals received by the second sensor 114 to determine BP values of the user 110. The first user device 102a (or the client application 112) may then direct data, signals, and/or BP values associated with the second sensor 114 (or the second user device 102b) to the application server 124 via the network 122. In some examples, the second sensor 114 (or the second user device 102b) is configured to communicate directly with the application server 124 (e.g., via the network 122). In some examples, the second user device 102b is configured to communicate (e.g., transmit BP data) with the first user device 102a and/or the application server 124 using blockchain technology. This use of blockchain technology may be useful to build trust with users, encouraging engagement with the system 100 and effective BP monitoring and management.

It should be appreciated that the client application 112 may be configured to run (or operate) on the second user device 102b. In such cases, the client application 112 may run on both the first and second user devices 102a, 102b or just the second user device 102b. Likewise, while not shown, the client application 112 may be configured to run (or operate) on other devices (e.g., a personal computer, tablet, etc.). In some examples, the client application 112 is independent of any specific device and is accessed via a web browser. For example, if the first user device 102a is a public device (or a third party device) and the second user device 102b is a wearable device without a screen, the user 110 may access the client application 112 via a web browser on their personal computer or another device (e.g., smartphone, tablet, etc.).

Means B (i.e., the second sensor 114) facilitates advanced monitoring for users identified as at-risk by Means A (i.e., the first sensor 104), enhancing the accuracy of diagnosis and enabling personalized management strategies. Means A functions as a mechanism to qualify users and patients for the use of Means B. By providing initial BP measurements across a broad user base, Means A identifies individuals who may be at risk of hypertension or related health conditions. This initial screening process is critical, as it filters and funnels qualified users into the next phase of monitoring with Means B. Means A effectively stratifies the population by assessing BP levels and other relevant health indicators, ensuring that those who require more advanced, continuous monitoring are promptly identified and guided toward using Means B. This seamless transition from widespread screening to targeted monitoring enhances early detection, accurate diagnosis, and personalized management of BP conditions across large populations.

Traditionally, a spot-check BP measurement (e.g., like given by the Means A sensor 104) is used for diagnosis of BP. For such traditional measurements, only 2 readings of BP are needed for a diagnosis, which often leads to inaccurate diagnosis due to the limited sample size. As such, the system 100 may be operated such that Means A is used to screen for possible HTN (but not diagnose) and Means B is used to get continuous data to make a clear (or final) diagnosis. As such, the diagnosis of HTN is based on continuous data, rather than episodic data. In some examples, the first sensor 104 measures BP in mmHg, performing spot-check measurements, and screening users for the second sensor 114. The second sensor 114, being of a different type and adapted for continuous or semi-continuous measurements, may measure BP using advanced metrics such as Time-in-Target Range (TTR), Hi-Load, and Cumulative Blood Pressure Load (CBPL), each of which is to be described in greater detail herein. By utilizing these advanced metrics, the second sensor 114 classifies users according to risk profiles and provides personalized recommendations, enhancing the diagnosis and management of HTN. In some examples, by presenting these advanced metrics, the system 100 enhances user engagement and compliance in managing BP conditions.

In some examples, the system 100 employs artificial intelligence (AI) predictive modeling to classify users according to risk profiles. For example, the continuous BP data collected by the second user device 102b may be processed and/or analyzed by an AI model to classify users according to risk profiles. In such examples, the AI model may provide personalized recommendations for managing BP conditions. By analyzing continuous BP data, the system 100 enhances early detection and intervention.

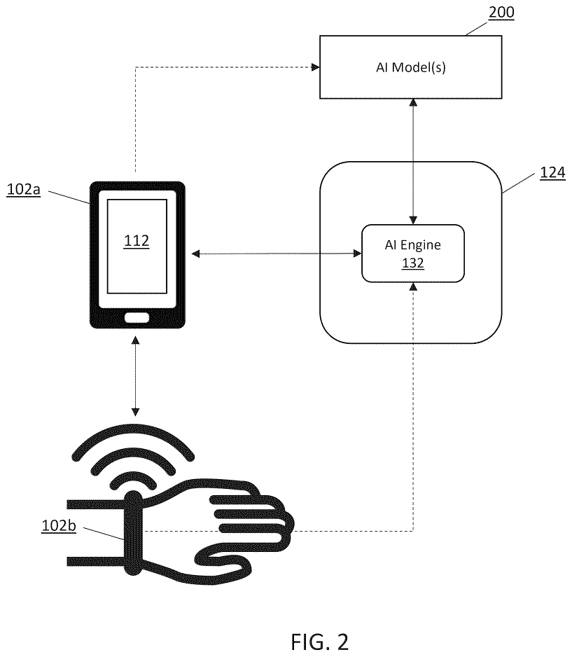

As shown in FIG. 2, the AI engine 132 of the application server 124 may interface with at least one AI model 200. In some examples, data from the second user device 102b (or second sensor 114) is provided to the AI model 200 via the client application 112 of the first user device 102a. The client application 112 may provide the data to the AI model 200 via the AI engine 132 of the application server 124. In some examples, the client application 112 (or the first user device 102a) is configured to communicate with the AI model 200 directly (e.g., over a network connection). The client application 112 may provide the data to the AI model 200 via the AI engine 132 of the application server 124. In some examples, the second user device 102b (or second sensor 114) is configured to communicate with the AI model 200 via the AI engine 132 (i.e., bypassing the first user device 102a and the client application 112). In some examples, the AI model 200 is a large language model (LLM). In some examples, the AI model 200 is an internal model that runs on the application server 124. In some examples, the AI model 200 is an external model that the AI engine 132 communicates with via one or more APIs. In some examples, the AI model 200 is a foundational model. In some examples, the AI model 200 is a specialized model that is trained solely for use with the system 100.

In some examples, the AI model 200 is a specialized model trained to perform a diagnosis based on the BP data collected by the second user device 102b. In some examples, the AI model 200 is a specialized model trained to plan and track the intervention tailored for the user 110. In such examples, the AI model 200 can outperform deterministic pre-programmed algorithms by learning from user dynamics or dynamics from other users (e.g., having with similar characteristics as the user 110). In some examples, the AI model 200 is iteratively trained to improve the accuracy of the model outputs. For example, the AI model 200 may be trained using a first data set corresponding to a first set of continuous BP data and retrained using a second data set corresponding to a second set of continuous BP data. In some examples, the AI model 200 may be trained using a first data set corresponding to the BP data collected by the first user device 102a. Predictions by the AI model 200 from the first data set (e.g., diagnosis, intervention efficacy, etc.) may be validated using the second data set corresponding to the continuous or semi-continuous BP data collected by the second user device 102b. Such training techniques enhance the ability of the AI model 200 to accurately screen and diagnosis users based BP data from the first and second user devices 102a, 102b.

In addition to its continuous and automated monitoring capabilities, Means B (i.e., the second user device 102a or second sensor 114) actively reinforces the user's engagement with their own health monitoring and awareness of their medical condition. By exposing the user 110 to continuous data, it empowers them to become more informed about their BP patterns and overall cardiovascular health. This constant flow of information allows both the system 100 and the user 110 to optimize interventions that are most effective for them personally. The user can see real-time feedback on how lifestyle changes or treatments impact their BP, which provides positive reinforcement and encourages adherence to their health management plan. This positive feedback loop maximizes engagement, as users are motivated by the tangible improvements they observe. Means B not only delivers advanced data but also fosters a proactive approach to health management, enabling users to take an active role in optimizing their health outcomes through sustained engagement and personalized interventions. In some examples, Means B (i.e., the second user device 102b or second sensor 114) is adapted to provide personalized recommendations, incorporating gamification elements to motivate users. By engaging users through rewards and progress tracking, the system enhances adherence to BP management plans.

In some examples, the system 100 provides adaptive screening for personalization and compliance. In such examples, the first user device 102a not only measures BP but also adapts the user interface (UI) of the client application 112 to the user's rationale for using the system 100, facilitating personalized but widespread screening. By tailoring the screening protocols and UI according to different user personas, the system 100 enhances user engagement and effectively screens users for candidates to use a second user device 102b (or second sensor 114). In some examples, the first user device 102a facilitates the user 110 ordering the second user device 102b (or the second sensor 114), streamlining the transition to advanced monitoring. In some examples, the first user device 102a and/or the second user device 102b can prompt users to refer relatives or friends to use the first user device 102a (or first sensor 104) for screening, enhancing early detection efforts. In such examples, the first sensor 104 facilitates spot-check BP measurements and screens new users for the second sensor 114.

In some examples, the system 100 supports integration with electronic health records (EHRs) and/or external data sources. For example, the first user device 102a, the second user device 102b, and/or the application server 124 are configured to access EHRs stored in an external EHR database 140. The EHR database 140 may be a cloud-based database accessible via the network 122. In some examples, the EHR database 140 is affiliated with a medical institution, a medical provider, an insurance provider, a university, or another entity that creates and/or manages EHRs. In some examples, the EHRs of the user 110 are stored in the user data database 136 by the user engine 128 of the application server 124. Similarly, the first user device 102a, the second user device 102b, and/or the application server 124 may be configured to access data from at least one external data source 142. In some examples, the external data source 142 is accessed by the system 100 via an API. The external data source 142 may be a cloud-based database accessible via the network 122. In some examples, the external data source 142 is a medical record portal, a medical insurance portal, a fitness tracker, a wearable device data manager, a food tracker, a fitness application, a health hub, or any other suitable data source. Examples of external data sources include Apple Health, Google Fit, Fitbit, Samsung Health, MyFitnessPal, Strava, Garmin Connect, Oura, Whoop, FatSecret, Withings Health Mate, Runkeeper, MapMyFitness, Endomondo, and Runtastic. In some examples, data from the external data source 142 is periodically captured and stored/updated in the user data database 136 by the user engine 128 of the application server 124.

In some examples, the continuous or semi-continuous monitoring data collected by the second user device 102b (or second sensor 114) is used to classify the user 110 according to risk profiles, more accurate diagnoses, and indications of other BP-related diseases by integrating data associated with the user 110 from EHRs, insurance data, genomic data, lab and imaging data, etc., retrieved from the EHR database 140 and/or the external data source 142. By combining the continuous or semi-continuous BP measurements recorded by the second sensor 114 with comprehensive health records, the system 100 provides personalized recommendations for managing BP conditions and enhances the accuracy of risk assessment of BP, HTN, and related disorders.

In some examples, the first user device 102a (or first sensor 104) and/or the second user device 102b (or second sensor 114) is configured to communicate with at least one additional device 144. In some examples, the additional device 144 is a continuous electrocardiogram (ECG) monitoring device. In such examples, the system 100 may provide integrated continuous ECG monitoring to enhance diagnostic capabilities. By combining BP data with ECG data, the system 100 can classify users according to risk profiles, more accurate diagnoses, and indications of other BP-related diseases, and provide personalized recommendations, improving the management of BP and cardiovascular conditions.

In some examples, the system 100 supports integration with sleep apnea applications and sleep tracking systems. For example, the additional device 144 in communication with the second user device 102b may be a sleep tracking system, sensor, apparatus, or device. In some examples, the first user device 102a, the second user device 102b, and/or the application server 124 are configured to access sleep apnea or sleep tracking data from the external data source 142. By integrating sleep apnea assessments and sleep data, the continuous or semi-continuous monitoring data collected by the second user device 102b (or second sensor 114) can be used to classify users according to risk profiles, such as a likelihood of sleep apnea or efficacy of sleep apnea treatments, and provides personalized recommendations, addressing both BP conditions and sleep disorders.

In some examples, the system 100 supports integration with Continuous Glucose Monitoring (CGM) systems that track real-time glucose levels, enabling the system 100 to correlate BP measurements with glycemic fluctuations. Examples of such CGM systems include Dexcom G6, Dexcom G7, FreeStyle Libre 2, FreeStyle Libre 3, Medtronic Guardian Connect, and Eversense XL. CGM integration provides insights into the interplay between glucose control and BP variations, enabling personalized recommendations for managing BP conditions. Example recommendations include dietary adjustments, changes in medication timing, or exercise plans tailored to the user's glucose and BP patterns, improving overall cardiovascular and metabolic health.

In some examples, the continuous or semi-continuous monitoring data collected by the second user device 102b (or second sensor 114) is adapted to provide personalized recommendations to the user 110 for managing a BP condition, including integration with medication management systems. Such medication management systems may include medication apps, smart pill bottles, or health-system compliance reminders. By tracking medication adherence and correlating it with BP measurements, the system 100 supports effective BP management.

The system 100 may be configured to support integration with fitness monitoring apps and systems. In some examples, the continuous or semi-continuous monitoring data collected by the second user device 102b (or second sensor 114) is integrated with fitness monitoring apps and systems. As described above, the external source 142 may be a fitness app. Likewise, the additional device 144 may be a fitness system. By integrating fitness monitoring apps and systems that monitor physical activity, and correlating it with BP measurements, the system 100 provides personalized physical recommendations for managing BP conditions.

Similarly, the system 100 may be configured to support integration with mindfulness monitoring apps and systems that monitor mindfulness and relaxation activities. In some examples, the continuous or semi-continuous monitoring data collected by the second user device 102b (or second sensor 114) is integrated with mindfulness monitoring apps and systems. Such mindfulness monitoring apps and systems may correspond to the external data source 142 and the additional device 144, respectively. By integrating mindfulness monitoring apps and systems that monitor mindfulness and relaxation activities, and correlating it with BP measurements, the system 100 can provide personalized relaxation recommendations for managing BP conditions.

In some examples, the additional device 144 is a cardiac device. The cardiac device may be a pacemaker, defibrillator, a left ventricular assist device (LVAD), or any other suitable device. By combining BP data with data from cardiac devices, the system 100 can classify users according to risk profiles, more accurate diagnoses, and indications of other BP-related diseases, and provide personalized recommendations, improving the management of BP and cardiovascular conditions.

In some examples, the system 100 is configured to retrieve environmental factors from the external data sources 142. By analyzing environmental factors (e.g., weather patterns, stress levels, travel, calendar integrations) and integrating them into risk profiles, the system 100 can tailor interventions to the user's context, improving BP condition management.

In some examples, the second user device 102b (or second sensor 114) provides features that facilitate social support by connecting users with support groups. For example, the continuous or semi-continuous monitoring data collected by the second user device 102b (or second sensor 114) may be used to identify a support group for the user 110. In some examples, the support group is selected based on BP metrics, conditions, or BP-related diseases associated with the user 110. In some examples, the user 110 interacts with the support group via the client application 112 of the first user device 102a. This use of support groups enhances adherence to BP management by leveraging community engagement. In some examples, the continuous or semi-continuous monitoring data collected by the second user device 102b (or second sensor 114) is used to facilitate virtual and/or remote coaching sessions, helping users manage their BP condition effectively. By classifying users according to risk profiles, the system 100 ensures that coaching is tailored to individual needs. In some examples, the second user device 102b (or second sensor 114) provides personalized recommendations or notifications to the user 110. Such notifications can include recommendations to see a physician with a means to schedule physician consultation (e.g., via the client application 112 of the first user device 102a). In some examples, the user 110 can adapt notification channels based on their own preferences, enhancing user engagement and management of BP conditions. For example, the user 110 may use the first or second user device 102a, 102b to configure the types of notifications they are interested in receiving and the medium for receiving notifications (e.g., via the first user device 102a, the second user device 102b, email, etc.).

### Therapeutic Metrics and Markers

In some embodiments, the system 100 is configured to calculate, in real-time, advanced therapeutic markers from the continuous or semi-continuous BP data collected via Means B (i.e., second user device 102b or second sensor 114). In some examples, the therapeutic markers quantify the duration and/or magnitude by which the user's BP exceeds or falls below a predetermined target range, providing a combined assessment of time and BP deviations. By receiving passive and automated BP readings, the system 100 calculates these markers, offering a more comprehensive understanding of BP abnormalities and associated health risks than traditional SBP and DBP measurements alone. This approach directly addresses the critical need to measure the extent and duration of exposure to unhealthy BP conditions, which are key determinants of end-organ damage and cardiovascular events.

By utilizing the advanced therapeutic markers derived from multiple BP measurements collected over an extended period using the second sensor 114, users experience BP monitoring that is significantly enhanced. The advanced therapeutic markers provided herein overcome notable limitations of traditional BP measurement methods. For example, traditional methods like Home Blood Pressure Monitoring (HBPM) or office BP measurements are infrequent and provide sparse, random BP values. Traditional methods like Ambulatory Blood Pressure Monitoring (ABPM) offer more frequent readings (e.g., approximately 40 measurements over 24 hours) but are typically limited to one or two days due to user discomfort from the cumbersome equipment. Consequently, the standard of care has been constrained to treating patients based on isolated BP values taken during office visits or short-term monitoring, such as whether they are higher or lower than a specific threshold (e.g., 140 mmHg) on the day of measurement.

Traditional BP measurement methods fail to measure and quantify the extent and duration by which an individual's BP deviates from the healthy target range over time. Traditional BP monitoring methods provide sporadic and isolated measurements, failing to capture the continuous exposure of end organs to unhealthy BP levels, which is crucial for assessing cardiovascular risk and organ damage. Knowing that a patient's BP was 160 mmHg during a doctor's visit or that their average systolic BP was 140 mmHg during a single night of ABPM does not provide sufficient insight into the chronic exposure of their organs to unhealthy BP levels. End organs such as the kidneys, brain, heart, and eyes suffer damage from continuous exposure to out-of-range BP conditions, particularly elevated BP, over the long term. It is the cumulative effect—the longer and further these organs are exposed to high BP—that determines the degree of end-organ damage and the likelihood of adverse cardiovascular events. As such, there is a pressing need to monitor BP continuously, or at least more frequently, to obtain a comprehensive picture of how far and for how long the body is exposed to unhealthy BP conditions. In some examples, the desired monitoring period is several days (e.g., at least three days). As described above, Means B (i.e., the second user device 102b or second sensor 114) provides the capability for extended BP monitoring using wearable devices capable of continuous or semi-continuous measurements. This approach surpasses the measurement experience of traditional BP measurement methods, such as ABPM, which is limited by user discomfort and practicality concerns over longer periods.

In addition to extended monitoring, there is a need to generate new metrics that accurately quantify the time and magnitude of exposure to unhealthy BP conditions. Traditional metrics, such as average BP values, fail to account for fluctuations and excursions that can significantly impact cardiovascular risk. A patient's BP may vary widely throughout the day, and relying solely on average values can obscure periods when BP is dangerously high or low.

In some examples, a Time-in-Target Range (TTR) metric is used to calculate, in real-time, the percentage of time the user's BP remains within a predetermined target range. TTR is measured over a specified monitoring period (at least three consecutive days). In some examples TTR is calculated by dividing the total time during which BP readings are within the target range by the total monitoring time, then multiplying by 100 to express the value as a percentage. For instance, if a user's BP stays within the target range for 60 out of 72 hours, their TTR is approximately 83%. The predetermined target range may modified for each user (e.g., by the user, by the user's doctor, automatically by the client application 112 or application server 124, etc.).

TTR provides a dynamic understanding of BP control by quantifying how consistently the BP stays within healthy limits (e.g., the target range). In some examples, periods when the BP is outside the target range are highlighted or flagged, emphasizing the duration of exposure to potentially harmful levels. A higher TTR indicates consistent BP control and is associated with a lower risk of end-organ damage and cardiovascular events. Clinicians may use TTR to assess the effectiveness of current treatment plans and to make informed decisions about medication adjustments or lifestyle interventions. Monitoring TTR helps identify periods when BP control is suboptimal, prompting timely interventions to improve overall cardiovascular health.

In some examples, a Hi-Load (HL), or Cumulative Blood Pressure Load (CBPL), marker determines the total burden of elevated BP over time by combining both the magnitude and duration of BP deviations from the target range. In some examples, HL is calculated by summing the products of the BP deviation from the target range and the time spent at each deviation level (e.g., mmHg×hours). For example, if a user's systolic BP is 10 mmHg above the target for 5 hours and 20 mmHg above for 2 hours, the HL would be: (10 mmHg×5 hours)+(20 mmHg×2 hours)=90 mmHg·hours. In some examples, HL is quantified using different units (e.g., mmHg×years).

HL offers insight into the chronic stress placed on the cardiovascular system by quantifying not just how often, but how severely BP exceeds or falls below healthy levels. It effectively measures the “area under the curve” where BP readings are outside the target range, providing a more comprehensive assessment of cardiovascular risk. A higher HL indicates greater exposure to harmful BP, correlating with increased risk of organ damage and cardiovascular events. Clinicians may use HL to tailor treatment strategies aimed at reducing both the frequency and severity of BP deviations, thereby lowering the cumulative cardiovascular risk.

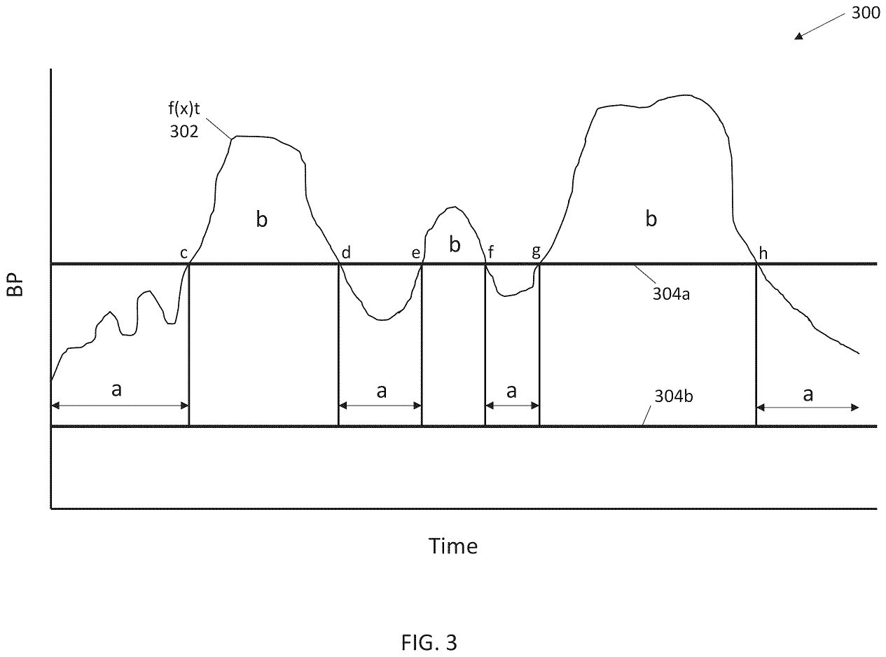

FIG. 3 is a graph 300 illustrating an example BP measurement in accordance with aspects described herein. As shown, the graph 300 includes a BP signal f(x)t 302. In some examples, the BP signal f(x)t 302 corresponds to a continuous BP measurement collected via Means B (i.e., the second user device 102b or second sensor 114). The x-axis of the graph 300 represents time (e.g., in units of seconds) and the y-axis of the graph 300 represents BP (e.g., in units of mmHg). The thresholds (or boundaries) 304a, 304b represent the target range that is used to determine the TTR of the user 110. In other words, the BP of the user 110 is considered to be “in-range” when the BP signal f(x)t 302 is less than the upper threshold 304a and greater than the lower threshold 304b. As such, the periods labeled ‘a’ correspond to the user's TTR. In some examples, the TTR is calculated as a percentage of the total measurement time. For example, if the ‘a’ periods make up 40% of the total measurement time, then the TTR of the user 110 may be 40%.

In some examples, the HL of the user 110 is calculated by measuring the “area under the curve” when the BP signal f(x)t 302 is over the upper threshold 304a or below the lower threshold 304b. The regions labeled ‘b’ correspond to the elevated BP regions that contribute to the user's HL. The total region ‘b’ from FIG. 3 can be calculated using Equation (1) below:

$$b = {\int }_{d}^{c} f ( x ) t + {\int }_{f}^{e} f ( x ) t + {\int }_{h}^{g} f ( x ) t . \qquad Equation ( 1 )$$

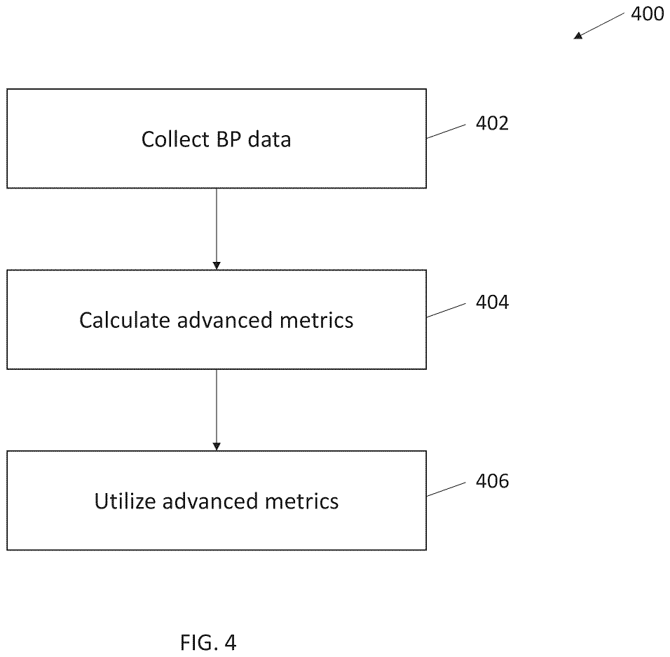

FIG. 4 is a flowchart of a method 400 for calculating and utilizing advanced BP metrics in accordance with aspects described herein. In some examples, the method 400 is configured to be performed using the system 100.

At step 402, the system 100 receives multiple BP measurements from the user 110. In some examples, the BP measurements are received continuously or semi-continuously over a time period (e.g., at least three consecutive days). The BP measurements are obtained using the second user device 102b (or second sensor 114). In some examples, the second user device 102b is a wearable device that performs continuous or semi-continuous monitoring without requiring user initiation. The extended monitoring period allows for the capture of BP fluctuations and patterns that are not detectable with shorter-term monitoring methods. In other examples, the BP measurements may be received from a combination of the first and second user devices 102a, 102b (or just the first user device 102a). In some examples, the BP measurements are received from a combination of the first user device 102a, the second user device 102b, and/or the additional device(s) 144. In some cases, receiving BP measurements from multiple devices is advantageous as it increases the density of BP points collected from the user.

At step 404, the system 100 calculates the TTR and HL values for the received BP measurements. In some examples, the metric engine 130 of the application server 124 is configured to calculate the TTR and HL values. As described above, these metrics quantify the extent and duration of BP deviations from the target range, providing a detailed profile of the user's BP control. By focusing on both the time spent outside the healthy range and the magnitude of the deviations, the system 100 can address the critical factors that contribute to end-organ damage and cardiovascular events.

At step 406, the calculated TTR and HL values are presented to the user 110 through a graphical user interface (e.g., of the client application 112), enhancing their awareness of their BP patterns. The system 100 may also provide personalized recommendations based on these metrics, including advice on lifestyle modifications, medication adjustments, and other interventions aimed at improving BP control. Examples of such recommendations include instructions to: reduce salt intake, increase potassium intake (e.g., through fruits and vegetables), follow Dietary Approaches to Stop Hypertension (DASH), maintain a healthy weight, lose an amount of weight, exercise regularly (e.g., aerobic exercises, walking, cycling, etc.), limit alcohol consumption, quit smoking, reduce caffeine intake, practice stress management techniques (e.g., mediation, yoga, etc.), get adequate sleep (e.g., 7-9 hours per night), incorporate breathing exercises, consume omega-3 fatty acids (e.g., fish or flaxseeds), limit intake of processed foods or high-sugar foods, maintain or improve hydration, incorporate mindfulness or relaxation practices, increase daily fiber intake, reduce consumption of red or processed meat, avoid sedentary behavior and increase daily movement, moderate red wine consumption, and consume dark chocolate or cocoa products in moderation. By tracking these metrics over time, users and healthcare providers can assess the effectiveness of interventions and make informed decisions to optimize management strategies.

The method 400 moves beyond traditional BP measurements by incorporating advanced therapeutic markers, offering a more comprehensive and accurate assessment of cardiovascular risk. Traditional BP measurement techniques do not achieve the same level of detailed analysis, as they often rely on infrequent measurements that do not capture the full extent of BP variability and its impact on end organs. By extending the monitoring period and introducing metrics that quantify both the duration and magnitude of BP deviations, the method 400 provides critical insights that are essential for effective HTN management.

As discussed above, the system 100 improves the monitoring and management of HTN by leveraging detailed temporal BP data to inform treatment decisions. The system 100 addresses the unmet need for extended and frequent BP monitoring and introduces new metrics that directly correlate with the physiological factors that lead to end-organ damage and cardiovascular events. The system 100 collects multiple BP measurements over an extended period (e.g., at least three consecutive days) using continuous or semi-continuous monitoring devices (i.e., the second user device 102b or second sensor 114). The system 100 calculates therapeutic BP markers (e.g., TTR and HL) that quantify the time and magnitude of BP deviations from a predetermined target range. The system 100 utilizes these metrics (or markers) to provide a comprehensive assessment of BP control, inform personalized management strategies, and improve patient outcomes by reducing the risk of end-organ damage. By implementing the method 400, healthcare providers can move beyond treating patients based solely on isolated BP values and instead focus on the factors that truly matter for long-term health. This approach represents a significant advancement in HTN management, offering the potential to reduce the incidence of cardiovascular events and improve quality of life for individuals with elevated BP.

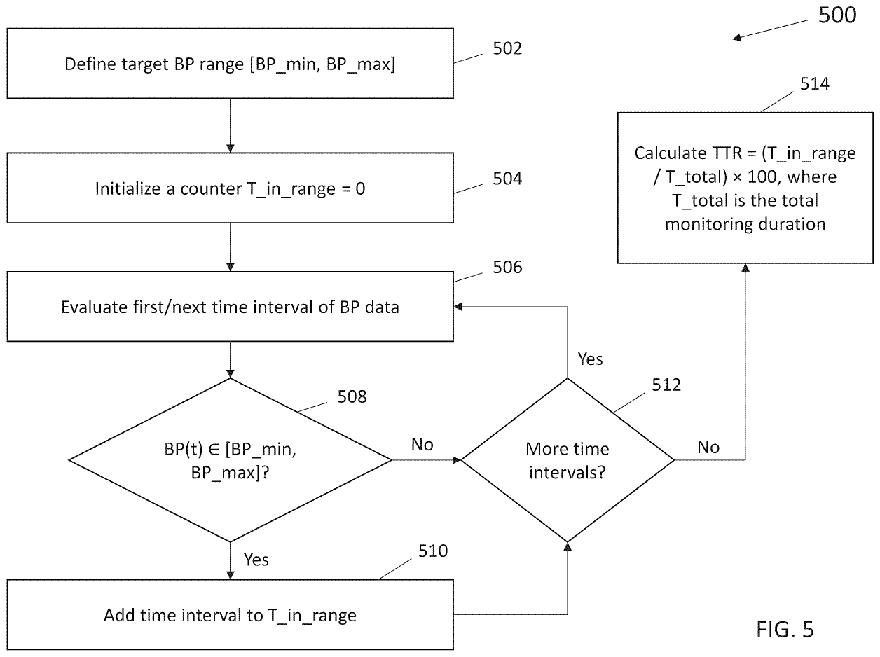

FIG. 5 is a flowchart of a method 500 for calculating TTR in accordance with aspects described herein. In some examples, the method 500 is configured to be performed by the metric engine 130 of the application server 124. In some examples, the method 500 is performed during step 404 of the method 400.

At step 502, the metric engine 130 defines the target BP range for the user 110. In some examples, the target BP range includes a BP_max value (i.e., the upper threshold 304a) and a BP_min value (i.e., the lower threshold 304b). As described above, the target BP range may be may modified for each user (e.g., by the metric engine 130, by the user, by the user's doctor, by the client application 112, etc.).

At step 504, the metric engine 130 initializes a counter T_in_range to 0. In some examples, the counter T_in_range is initialized before each new TTR measurement.

At step 506, the metric engine 130 receives the BP data of the user 110 (e.g., BP signal f(x)t 302) and evaluates the first time interval dt of the BP data. In some examples, the time interval dt corresponds to a sampling rate (or frequency) of the BP data. In some examples, the time interval dt corresponds to a predetermined time interval (e.g., 1 ms, 1 sec, etc.).

At step 508, the metric engine 130 determines whether the first time interval data BP(t) falls within the target BP range (i.e., is greater than BP_min and less than BP_max). If so, the method 500 proceeds to step 510. Otherwise, the method 500 proceeds to step 512.

At step 510, in response to a determination that the first time interval data BP(t) falls within the target BP range, the metric engine 130 adds the time interval to the counter T_in_range. In some examples, the length of the time interval is added to the counter. In some examples, the counter is incremented by a fixed increment (e.g., +1).

At step 512, the metric engine 130 determines whether there are more time intervals dt of the BP data to evaluate. In other words, the metric engine 130 determines whether the complete BP signal has been processed, indicating the end of the TTR measurement period. If so, the method proceeds to step 514. Otherwise, the method returns to step 506 and repeats for each time interval dt until the complete BP signal has been processed (e.g., by steps 508 and 510).

At step 514, the metric engine 130 calculates the corresponding TTR value. In some examples, Equation (2) below is used to calculate the TTR value:

$$TTR = ( T\_in \_range / T\_total ) \times 100. \qquad Equation ( 2 )$$

In Equation (2), T_total represents the total monitoring duration (i.e., the combined length of the time intervals dt).

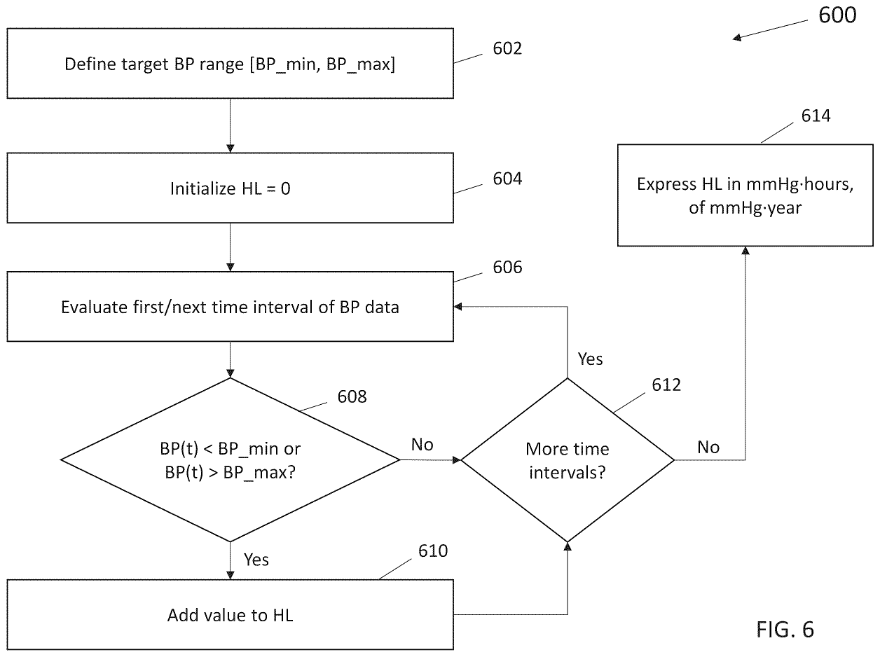

FIG. 6 is a flowchart of a method 600 for calculating HL in accordance with aspects described herein. In some examples, the method 600 is configured to be performed by the metric engine 130 of the application server 124. In some examples, the method 600 is performed during step 404 of the method 400.

At step 602, the metric engine 130 defines the target BP range for the user 110. In some examples, the target BP range includes a BP_max value (i.e., the upper threshold 304a) and a BP_min value (i.e., the lower threshold 304b). As described above, the target BP range may be may modified for each user (e.g., by the metric engine 130, by the user, by the user's doctor, by the client application 112, etc.).

At step 604, the metric engine 130 initializes the HL value to 0. In some examples, the HL value is initialized before each new HL measurement.

At step 606, the metric engine 130 receives the BP data of the user 110 (e.g., BP signal f(x)t 302) and evaluates the first time interval dt of the BP data. In some examples, the time interval dt corresponds to a sampling rate (or frequency) of the BP data. In some examples, the time interval dt corresponds to a predetermined time interval (e.g., 1 ms, 1 sec, etc.).

At step 608, the metric engine 130 determines whether the first time interval data BP(t) falls outside the target BP range (i.e., is less than BP_min or greater than BP_max). If so, the method 600 proceeds to step 610. Otherwise, the method 600 proceeds to step 612.

At step 610, in response to a determination that the first time interval data BP(t) falls outside the target BP range, the metric engine 130 adds a corresponding value to the HL value. In some examples, the value added to the HL value represents both the magnitude and the duration of the deviation from the target BP range. For example, if BP(t)>BP_max, the added value may correspond to (BP(t)−BP_max)×dt, where dt represents the length of the time interval. Likewise, if BP(t)<BP_min, the added value may correspond to (BP_min−BP(t))×dt.

At step 612, the metric engine 130 determines whether there are more time intervals dt of the BP data to evaluate. In other words, the metric engine 130 determines whether the complete BP signal has been processed, indicating the end of the HL measurement period. If so, the method proceeds to step 614. Otherwise, the method returns to step 606 and repeats for each time interval dt until the complete BP signal has been processed (e.g., by steps 608 and 610).

At step 614, the metric engine 130 expresses the corresponding HL value in units of mmHg·time (e.g., mmHg·hours, mmHg·days, mmHg·weeks, or mmHg·year).

It should be appreciated that the advanced BP metrics (or markers) may be utilized in combination with the system 100 in a variety of applications and use cases. For example, multiple BP measurements may be received from a wearable sensor (e.g., the second user device 102b or second sensor 114) worn by the user 110 continuously over a month. The wearable sensor provides continuous or semi-continuous measurements, capturing the user's BP fluctuations throughout daily activities and rest periods. Using these multiple BP measurements, the system 100 determines both the TTR, HL, and CBPL. The calculated values of these metrics are then presented to the user via the client application 112, allowing the user 110 to visualize their BP control over the past month. Based on these values, the system 100 provides recommended actions such as risk assessments, lifestyle adjustments, or medication consultations to improve the user's BP management. In some examples, the therapeutic engine 134 monitors the subsequent efficacy of these actions with respect to changes in the TTR, HL, and/or CBPL.

In another example application of the system 100, multiple BP measurements are received from a wearable sensor (e.g., the second user device 102b or second sensor 114) that the user 110 wears intermittently over multiple years. The sensor captures semi-continuous measurements during periods when the user is at risk, such as during high-stress times or when engaging in physical activity. By aggregating this long-term data, the system 100 determines the user's HL and/or CBPL, providing insight into the cumulative impact of BP on their cardiovascular health. The HL/CBPL value is presented to the user 110 via the client application 112, and recommended actions are provided by the therapeutic engine 134, such as scheduling regular check-ups or adopting long-term lifestyle changes to mitigate risks associated with sustained elevated BP.

In some examples, multiple BP measurements are received from a wearable sensor (e.g., the second user device 102b or second sensor 114) over the course of a single day. The sensor provides continuous measurements, capturing BP changes in response to daily activities, meals, and stressors. The system 100 determines the TTR for that day and presents the TTR value to the user 110 at the end of the day (e.g., via the client application 112). Based on the day's TTR, the therapeutic engine 134 offers immediate recommended actions, such as relaxation techniques if BP was frequently above target ranges, helping the user 110 make real-time adjustments to manage their BP effectively. Similarly, the user 110 may wear a BP monitoring device that provides continuous or semi-continuous measurements over a week (e.g., the second user device 102b or second sensor 114). Multiple BP measurements are received and used to determine TTR, HL, and CBPL. By presenting these values to the user 110 and their healthcare provider, the therapeutic engine 134 can assess the efficacy of prescribed antihypertensive medications. The therapeutic engine 134 may recommend actions that include adjusting medication dosages or switching to alternative therapies based on the TTR, HL, or CBPL outcomes, optimizing the user's treatment plan.

In another example application of the system 100, multiple BP measurements from the wearable sensor over a month, with the sensor providing semi-continuous measurements during waking hours. The system 100 calculates the HL and/or CBPL to understand the overall BP burden. The HL and/or CBPL value is presented to the user 110 through the client application 112, highlighting periods of elevated BP. The therapeutic engine 134 may provide recommended actions such as dietary changes, increased physical activity, or stress management techniques tailored to the user's BP patterns to reduce their HL and/or CBPL in the following month. Effects of interventions may be presented in the context of HL and CBPL as well as traditional BP metrics.

In some examples, multiple BP measurements are collected from the wearable sensor worn periodically or continuously throughout a year. The measurements are continuous or semi-continuous when the sensor is worn. The system 100 determines TTR, HL, and CBPL over the year, providing a long-term view of the user's BP control. These values are presented to the user 110 and their healthcare provider to assess the quality of BP control, effect of management strategies, and the risk of developing future hypertension-related complications. The therapeutic engine 134 may provide recommended actions that include initiating or modifying long-term interventions, such as enrolling in a hypertension management program or adjusting lifestyle habits to improve BP control.

In another example application of the system 100, multiple BP measurements from various sources over a selected time period, such as a month. The various sources may include a wearable sensor (e.g., the second user device 102b or second sensor 114), the external data source(s) 142 (e.g., clinic readings), and the additional device(s) 144 (e.g., home BP monitors). The aggregated data, comprising continuous and spot-check measurements, is used to determine the user's TTR, HL, and CBPL. By presenting these comprehensive metrics to the user 110, the system 100 provides a holistic view of BP control across different environments and modalities. The therapeutic engine 134 may recommend actions generated based on this integrated data, offering personalized strategies to manage BP effectively in all aspects of the user's life.

In some examples, multiple BP measurements are received from a wearable sensor providing continuous monitoring over a selected time period. The system 100 calculates the TTR and presents it to the user 110 via the client application 112. If the TTR falls below a predetermined threshold, indicating that the user's BP is frequently outside the target range, the system 100 sends an alert and provides recommended actions (e.g., via the client application 112). These actions might include contacting a healthcare provider or adjusting medication under medical supervision, enabling timely interventions to prevent adverse health outcomes.

In another example application of the system 100, the advanced BP metrics are used to target users with known cardiovascular risk factors. Multiple BP measurements are received from a wearable sensor over a week or month, with continuous or semi-continuous data collection. The HL and/or CBPL is determined and presented to the user and their care team. Given the high-risk status, the therapeutic engine 134 provides recommended actions focused on intensive management strategies, such as immediate medical evaluation, aggressive lifestyle modifications, or medication adjustments to reduce the HL and/or CBPL and mitigate cardiovascular risks.

In some examples, the wearable sensor specifically monitors BP during sleep over several nights, providing continuous measurements. The system 100 determines the TTR during sleep and presents this nocturnal TTR to the user 110. If the TTR indicates frequent nocturnal hypertension, the therapeutic engine 134 provides recommended actions, such as discussing potential causes with a healthcare provider or evaluating for conditions like sleep apnea, thereby addressing issues that may not be evident during daytime BP monitoring.

In another example application of the system 100, the system 100 receives multiple BP measurements from the wearable sensor during and after exercise sessions over a selected time period. Continuous measurements capture BP changes and recovery rates of the user 110. The system 100 determines the TTR during post-exercise periods and presents this information to the user 110. Based on the TTR and recovery patterns, the therapeutic engine 134 recommends actions that may include adjusting exercise intensity, incorporating cooldown periods, or consulting a healthcare professional if BP remains elevated, promoting safe physical activity practices.

In some examples, multiple BP measurements are received from the wearable sensor during periods of identified stress over a week or month. The sensor provides continuous or semi-continuous measurements. The method calculates the HL and/or CBPL during these stress periods and presents the data to the user 110. Recognizing BP can be associated with stress, the therapeutic engine 134 provides recommended actions such as stress management techniques, mindfulness exercises, or counseling referrals to help the user 110 mitigate the impact of stress on their BP.

In another application of the system 100, multiple BP measurements are received from a wearable sensor over a month from a user with a non-traditional work schedules. The BP measurements with continuous monitoring across different shifts. The method determines the TTR during work and rest periods or HL and/or CBPL associated with specific shift patterns, and presents this information to the user 110. The therapeutic engine 134 Recommends actions that may include adjusting sleep hygiene or shift practices, dietary modifications, or scheduling regular BP assessments with healthcare providers to manage BP fluctuations associated with irregular schedules.

In some examples, multiple BP measurements are received from the wearable sensor over a selected time period, with continuous or semi-continuous data collection. The system 100 determines TTR, HL, and CBPL and presents these values to both the user 110 and their healthcare provider through a secure platform (e.g., the client application 112, external data source 142, EHR database 140, etc.). Based on the determined metrics, the healthcare provider can offer recommended actions, such as medication adjustments or scheduling follow-up appointments, facilitating collaborative BP management through remote monitoring.

In another application of the system 100, multiple BP measurements are collected from a wearable sensor continuously over a week. The system 100 calculates TTR and identifies patterns of BP elevation. The TTR value is presented to the user 110 with insights into specific times or activities associated with BP changes. The therapeutic engine 134 provides recommended actions tailored to these patterns, such as reducing sodium intake if BP spikes occur after meals, or encouraging breaks if BP rises during prolonged work periods, empowering the user 110 to make informed lifestyle adjustments.

In some examples, the system 100 is configured to provide integration with kidney disease or dialysis data platforms (e.g., as external data sources 142). The system 100 receives multiple BP measurements taken by the wearable sensor, and the calculation of TTR, HL, and CBPL are made. By considering kidney disease dialysis data alongside physiological data, the system 100 can predict the progression of kidney disease and can suggest interventions to lower the rate of kidney disease progression based on the TTR, HL, and/or CBPL. In this way, the system 100 provides a more comprehensive and accurate approach to kidney disease prevention.

In some examples, the system 100 is configured to make a diagnosis of HTN. Traditionally, a spot-check BP is used for diagnosis of HTN. The diagnosis is typically made from just two readings of BP, which leads to inaccuracies. Conversely, the system 100 may receive multiple BP measurements are from the wearable sensor and calculate TTR, HL, and CBPL. The system 100 (e.g., the therapeutic engine 134) may make a diagnosis of HTN based on these metrics from a wearable sensor.

The system 100 may be configured to monitor and manage BP for HTN in specific populations. In some examples, multiple BP measures are taken by a wearable sensor and BP metrics including TTR, HL, and CBPL are made by the system 100. Using these metrics, the system 100 can provide monitoring of people with or at risk for cognitive diseases, post-stroke, neurovascular diseases, sleep apnea (obstructive or central) retinal or ophthalmologic diseases related to BP, aortic aneurysms, aortic dissections, coronary artery disease, congestive heart failure, hypertensive cardiomyopathy, at risk for atrial fibrillation, kidney diseases, diabetes mellitus, during pregnancy, at risk for or people with pre-eclampsia, eclampsia, post-partum HTN, pheochromocytoma, resistant HTN, candidates for renal denervation, post-renal denervation, peripheral arterial disease, or erectile dysfunction.

In some examples, the management of BP and HTN, irrespective of diagnosis, disease state, or therapeutic area, is accomplished using the second user device 102b (or second sensor 114) and the advanced BP metrics described above. The success of interventions (e.g., lifestyle, procedural, device-related, or medications) may be measured via these same metrics. Furthermore, predictions based on the user's personal dataset in the context of an overall data library can be made, and suggestions of specific therapies, timing of therapies, dosing of therapies, decisions to continue or stop therapies are predicted rather than trial-and-error. In some examples, prediction of other diseases that are latent or undiagnosed can be made from the specific BP patterns and derived from the second user device 102b. For example, a pattern of continuous data from the second user device 102b (or second sensor 114) might suggest that the user 110 be screened for sleep apnea or kidney disease that they are previously unaware of.

In addition to the TTR, HL, and CBPL metrics/markers described above, the system 100 (or the metric engine 130) may be configured to calculate additional metrics or markers that provide improvements over traditional BP metrics. It should be appreciated that each of the following metrics/markers may be calculated during step 404 of the method 400.

In some examples, the system 100 is configured to calculate a Blood Pressure Severity Score (BPSS). The BPSS assigns weighted scores to BP readings based on the extent of deviation from the target range and the duration of each deviation. Greater deviations and longer durations receive higher weights. The cumulative score reflects the overall severity of BP irregularities over the monitoring period. BPSS provides a nuanced understanding of BP control by emphasizing significant deviations that pose higher risks. A higher BPSS suggests that the patient experiences severe and prolonged BP abnormalities. This information enables healthcare providers to prioritize interventions targeting the most critical periods of BP imbalance, potentially adjusting medications or recommending lifestyle changes to mitigate risks. In some examples, BPSS is represented in arbitrary units (e.g., a weighted score).

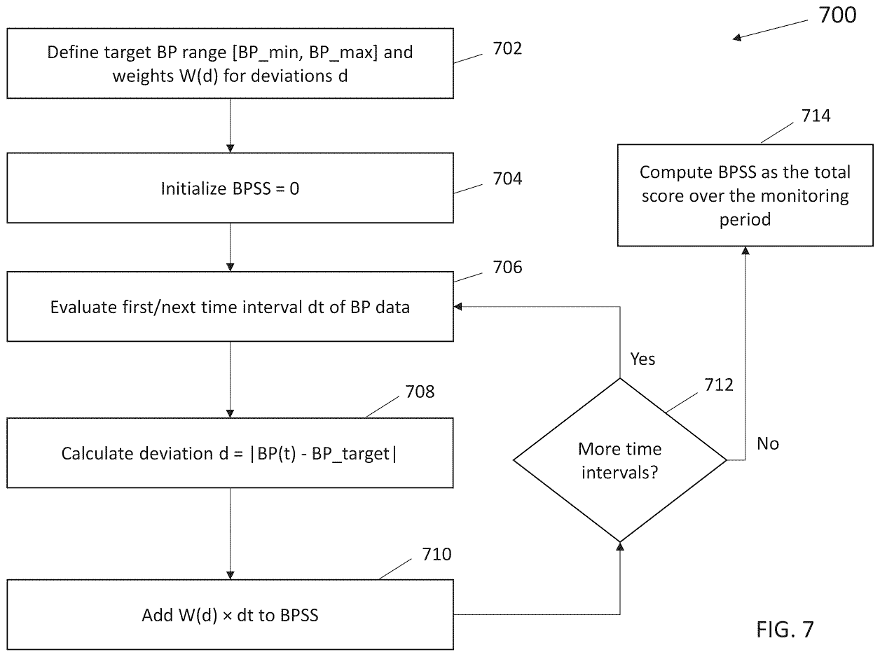

FIG. 7 is a flowchart of a method 700 for determining BPSS in accordance with aspects described herein. In some examples, the metric engine 130 is configured to perform the method 700, at least in part.

At step 702, the metric engine 130 defines the target BP range [BP_min, BP_max] and weights W(d) for deviations d. In some examples, W(d) is predefined as a linear or exponential weight function. In some examples, the weights W(d) are individualized for each user (e.g., based on characteristics, pre-existing health conditions, BP related diagnoses, etc.). In some examples, the same weights W(d) are used for each user.

At step 704, the metric engine 130 initializes BPSS=0. In some examples, BPSS is initialized each time BPSS is calculated.

At step 706, the metric engine 130 evaluates the first time interval dt of the BP data BP(t).

At step 708, the metric engine 130 calculates a deviation d for the first time interval dt. The deviation d may be calculated as d=|BP(t)−BP_target|. In some examples, BP_target is the midpoint of [BP_min, BP_max].

At step 710, the metric engine 130 adds W(d)×dt to BPSS.

At step 712, the metric engine 130 determines whether there are more time intervals dt of the BP data to evaluate. In other words, the metric engine 130 determines whether the complete BP(t) signal has been processed, indicating the end of the BPSS measurement period. If there are no more time intervals dt to evaluate, the method proceeds to step 714. Otherwise, the method returns to step 706 and repeats for each time interval dt until the complete BP signal has been processed (e.g., by steps 708 and 710).

At step 714, the metric engine 130 determines the value of BPSS for the measurement period (e.g., the length of BP(t)). In some examples, the metric engine 130 computes BPSS as the total score over the monitoring period.

In some examples, the system 100 is configured to calculate a Time-Weighted Average BP (TWABP) metric. TWABP represents the average BP over the monitoring period, with each BP reading weighted by the time spent at that level. The TWABP metric accounts for both the BP values and the duration at each value, offering an overall measure that emphasizes prolonged periods at specific BP levels. TWABP provides a more accurate reflection of the patient's typical BP exposure than simple arithmetic means. It highlights the impact of sustained BP levels on cardiovascular risk. Clinicians may use TWABP to assess the effectiveness of treatment regimens over time and to identify the need for interventions that address prolonged periods of uncontrolled BP. In some examples, TWABP is represented in units of mmHg.

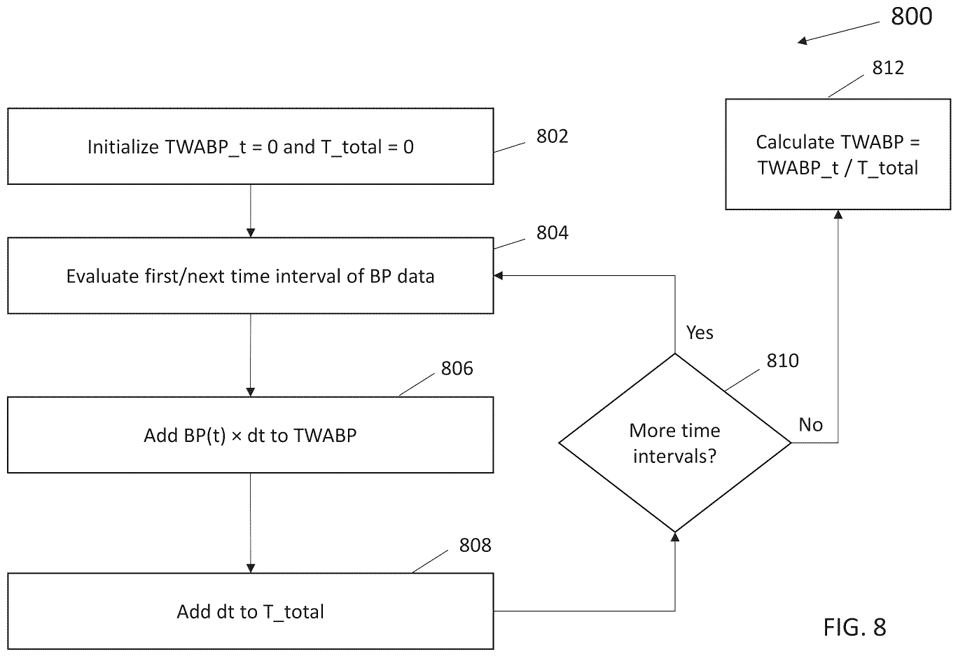

FIG. 8 is a flowchart of a method 800 for determining TWABP in accordance with aspects described herein. In some examples, the metric engine 130 is configured to perform the method 800, at least in part.

At step 802, the metric engine 130 initializes TWABP_t=0 and T_total=0. In some examples, TWABP_t and T_total are initialized each time TWABP is calculated.

At step 804, the metric engine 130 evaluates the first time interval dt of the BP data.

At step 806, the metric engine 130 adds BP(t)×dt to TWABP_t.

At step 808, the metric engine 130 adds dt to T_total. In other words, the length of dt is added to T_total.

At step 810, the metric engine 130 determines whether there are more time intervals dt of the BP data to evaluate. In other words, the metric engine 130 determines whether the complete BP(t) signal has been processed, indicating the end of the TWABP measurement period. If there are no more time intervals dt to evaluate, the method proceeds to step 812. Otherwise, the method returns to step 804 and repeats for each time interval dt until the complete BP signal has been processed (e.g., by steps 806 and 808).

At step 812, the metric engine 130 determines the value of TWABP for the measurement period (e.g., the length of BP(t)). In some examples, the metric engine 130 computes TWABP as TWABP=TWABP_t/T_total.

In some examples, the system 100 is configured to calculate a BP Deviation Index (BPDI) metric. BPDI measures the average magnitude of BP deviations from the target range, multiplied by the frequency of such deviations during the monitoring period. BPDI captures both how much and how often the BP deviates from the desired range. A higher BPDI indicates frequent and significant BP fluctuations, which can increase cardiovascular risk due to increased vascular stress. Identifying patients with a high BPDI allows clinicians to implement strategies that aim to stabilize BP, such as medication adjustments or lifestyle interventions focused on stress reduction and consistent medication adherence. In some examples, BPDI is represented in units of mmHg.

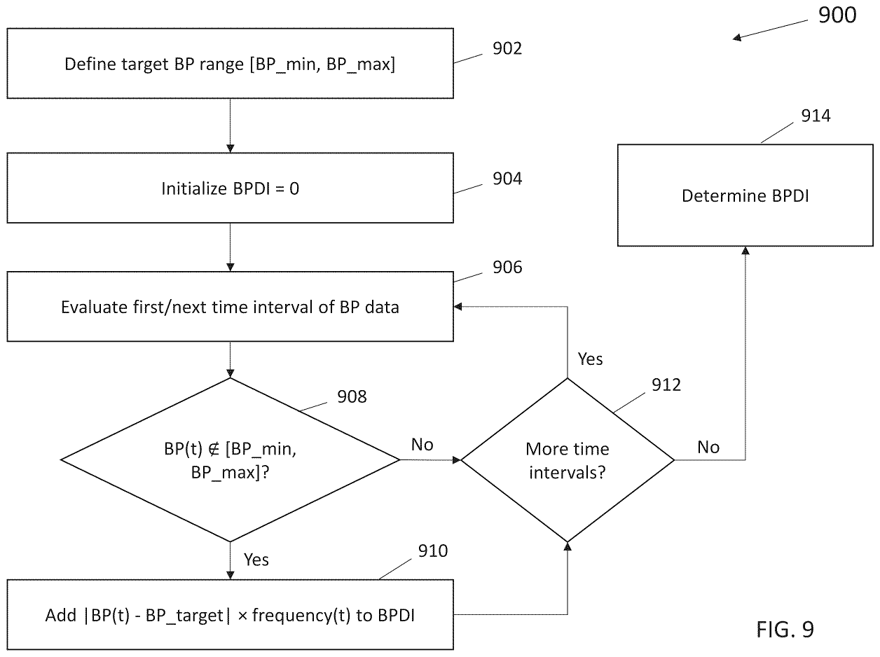

FIG. 9 is a flowchart of a method 900 for determining BPDI in accordance with aspects described herein. In some examples, the metric engine 130 is configured to perform the method 900, at least in part.

At step 902, the metric engine 130 defines the target BP range [BP_min, BP_max].

At step 904, the metric engine 130 initializes BPDI=0. In some examples, BPDI is initialized each time BPDI is calculated.

At step 906, the metric engine 130 evaluates the first time interval dt of the BP data.

At step 908, the metric engine 130 determines whether BP(t) V [BP_min, BP_max] during the first time interval dt. If so, the method proceeds to step 910. Otherwise, the method proceeds to step 912.

At step 910, in response to a determination that BP(t) V [BP_min, BP_max] during the first time interval dt, the metric engine 130 adds |BP(t)−BP_target|×frequency(t) to BPDI. Frequency(t) corresponds to the frequency of the BP signal BP(t) during the first time interval dt. In some examples, BP_target is the midpoint of the range [BP_min, BP_max].

At step 912, the metric engine 130 determines whether there are more time intervals dt of the BP data to evaluate. In other words, the metric engine 130 determines whether the complete BP(t) signal has been processed, indicating the end of the BPDI measurement period. If there are no more time intervals dt to evaluate, the method proceeds to step 914. Otherwise, the method returns to step 906 and repeats for each time interval dt until the complete BP signal has been processed (e.g., by steps 908 and 910).

At step 914, the metric engine 130 determines the value of BPDI for the measurement period (e.g., the length of BP(t)). In some examples, the metric engine 130 normalizes the value of BPDI by the total number of deviations (or total monitoring time).

In some examples, the system 100 is configured to calculate a Cumulative Hypertension Duration (CHD) metric. CHD sums the total time during which the user's BP exceeds a defined hypertension threshold (e.g., systolic BP>140 mmHg) over the monitoring period. CHD focuses exclusively on the duration of hypertensive episodes. CHD highlights the total exposure time to high BP levels, which is directly related to the risk of developing hypertensive complications. By quantifying this duration, healthcare providers can gauge the urgency of interventions needed to reduce hypertension exposure. In addition, reducing CHD through effective treatment can lower the risk of organ damage and cardiovascular events. In some examples, CHD is represented in units of hours or years.

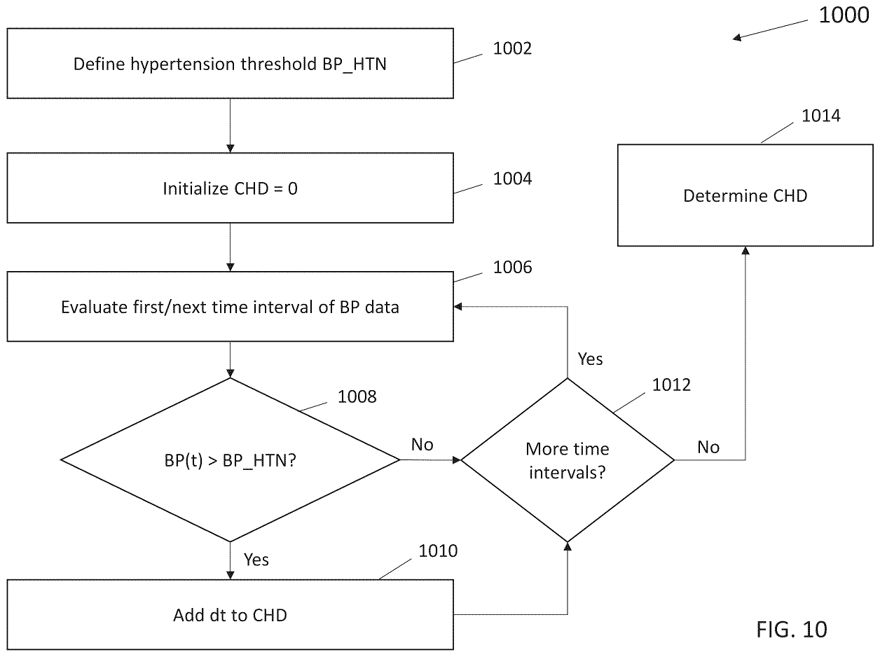

FIG. 10 is a flowchart of a method 1000 for determining CHD in accordance with aspects described herein. In some examples, the metric engine 130 is configured to perform the method 1000, at least in part.

At step 1002, the metric engine 130 defines a hypertension threshold BP_HTN (e.g., SBP>140 mmHg). In some examples, BP_HTN is individualized for each user (e.g., based on characteristics, pre-existing health conditions, BP related diagnoses, etc.). In some examples, the same BP_HTN is used for each user.

At step 1004, the metric engine 130 initializes CHD=0. In some examples, CHD is initialized each time CHD is calculated.

At step 1006, the metric engine 130 evaluates the first time interval dt of the BP data.

At step 1008, the metric engine 130 determines whether BP(t)>BP_HTN during the first time interval dt. If so, the method proceeds to step 1010. Otherwise, the method proceeds to step 1012.

At step 1010, in response to a determination that BP(t)>BP_HTN during the first time interval dt, the metric engine 130 adds dt to CHD. In other words, the length of dt is added to CHD.

At step 1012, the metric engine 130 determines whether there are more time intervals dt of the BP data to evaluate. In other words, the metric engine 130 determines whether the complete BP(t) signal has been processed, indicating the end of the CHD measurement period. If there are no more time intervals dt to evaluate, the method proceeds to step 1014. Otherwise, the method returns to step 1006 and repeats for each time interval dt until the complete BP signal has been processed (e.g., by steps 1008 and 1010).

At step 1014, the metric engine 130 determines the value of CHD for the measurement period (e.g. the length of BP(t)). In some examples, the CHD is expressed in hours or years.

In some examples, the system 100 is configured to calculate a BP Stability Coefficient (BPSC). The BPSC assesses the stability of BP by calculating the average change between consecutive BP readings over the monitoring period. Smaller average changes indicate more stable BP control, while larger changes suggest greater variability. High BP variability is associated with increased cardiovascular risk independent of average BP levels. A lower BPSC (i.e., indicating higher variability) may prompt clinicians to investigate underlying causes such as medication non-adherence or lifestyle factors. Interventions can then focus on promoting consistent BP control, potentially improving medication regimens or addressing behavioral factors contributing to variability. In some examples, the BPSC is represented in units of mmHg.

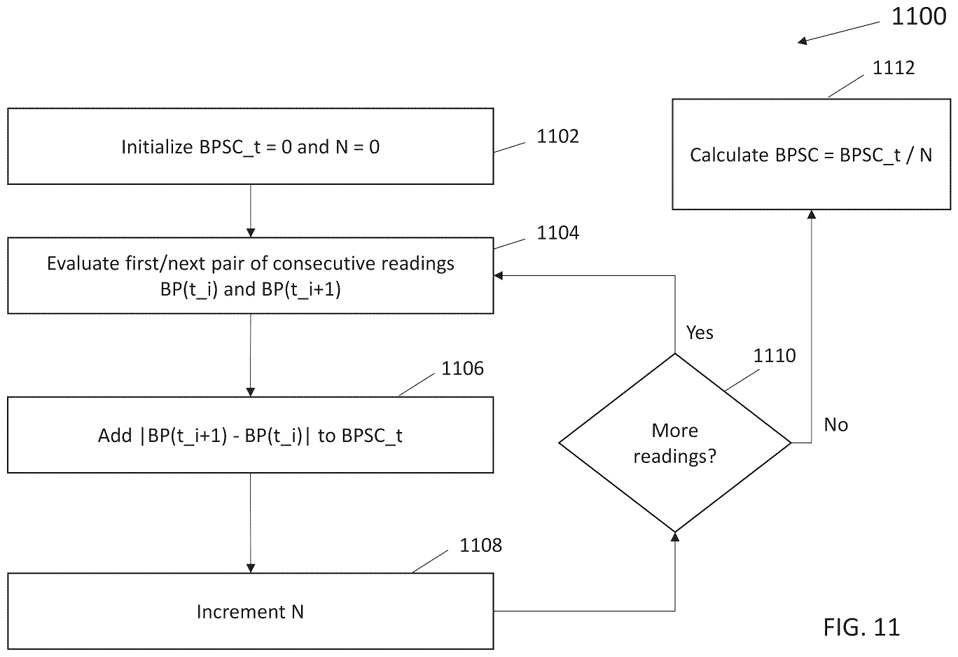

FIG. 11 is a flowchart of a method 1100 for determining BPSC in accordance with aspects described herein. In some examples, the metric engine 130 is configured to perform the method 1100, at least in part.

At step 1102, the metric engine 130 initializes BPSC_t=0 and N=0. In some examples, BPSC_t and N are both initialized each time BPSC is calculated.

At step 1104, the metric engine 130 evaluates a first pair of consecutive readings of the BP data. The consecutive readings are represented as BP(t_i) and BP(t_i+1). In some examples, the consecutive readings correspond to consecutive time intervals dt of the BP signal BP(t).

At step 1106, the metric engine 130 adds |BP(t_i+1)−BP(t_i)| to BPSC_t. In other words, the difference between the consecutive readings is added to BPSC_t.

At step 1108, the metric engine 130 increments N by 1.

At step 1110, the metric engine 130 determines whether there are more pairs of consecutive readings of the BP data to evaluate. In other words, the metric engine 130 determines whether the complete BP(t) signal has been processed, indicating the end of the BPSC measurement period. If there are no more pairs to evaluate, the method proceeds to step 1112. Otherwise, the method returns to step 1104 and repeats for each pair of consecutive readings.

At step 1114, the metric engine 130 determines the value of BPSC for the measurement period (e.g. the length of BP(t)). In some examples, the metric engine 130 computes BPSC as BPSC=BPSC_t/N.

In some examples, the system 100 is configured to calculate a Standard Deviation of BP (SD-BP). SD-BP measures the spread of BP values around their mean during a monitoring period. This metric provides a quantification of variability by assessing how much BP readings fluctuate. Higher SD-BP values indicate greater BP instability, which has been linked to increased cardiovascular risk and end-organ damage. Reducing BP variability is important in mitigating long-term risks associated with hypertension. Clinicians can use this metric to identify patients requiring interventions to stabilize BP, such as medication adjustments or lifestyle changes. In some examples, the SD-BP is represented in units of mmHg.

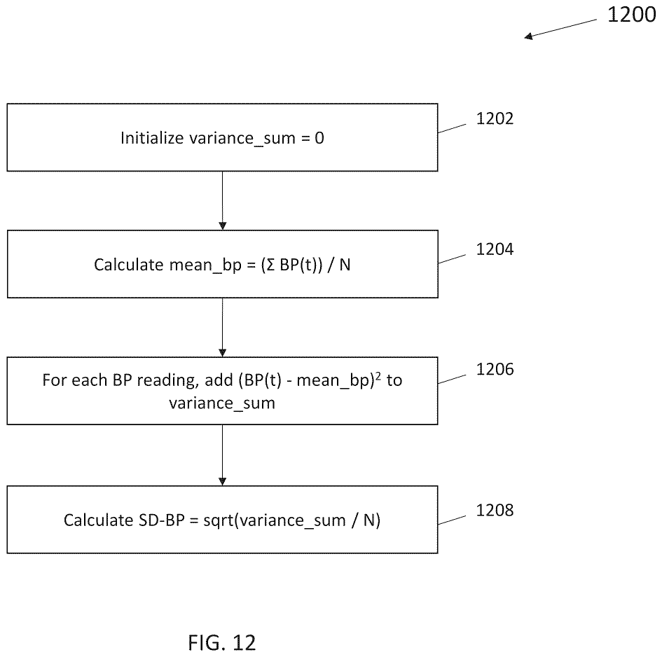

FIG. 12 is a flowchart of a method 1200 for determining SD-BP in accordance with aspects described herein. In some examples, the metric engine 130 is configured to perform the method 1200, at least in part.

At step 1202, the metric engine 130 initializes variance_sum=0. In some examples, variance_sum is initialized each time SD-BP is calculated.

At step 1204, the metric engine 130 calculates the mean BP of the BP data (i.e., the mean of BP signal BP(t)). In some examples, the metric engine 130 calculates the mean BP as mean_bp=(ΣBP(t))/N, where N is the total number of readings.

At step 1206, the metric engine 130 computes the variance of the BP data. In some examples, the metric engine 130 calculates a value (BP(t)−mean_bp)² for each BP reading (e.g., the value of BP(t) at each reading N) and adds the resulting values to variance_sum.

At step 1208, the metric engine 130 determines the value of SD-BP for the measurement period (e.g. the length of BP(t)). In some examples, the metric engine 130 computes SD-BP as SD-BP=sqrt(variance_sum/N).

In some examples, the system 100 is configured to calculate a Coefficient of Variation (CoV-BP). CoV-BP is a normalized measure of BP variability expressed as the ratio of the standard deviation to the mean BP value, multiplied by 100. It provides a percentage measure of variability relative to the mean BP. CoV-BP helps identify patients with disproportionate variability relative to their average BP levels. High CoV-BP values suggest significant BP instability, which increases the risk of cardiovascular events independent of mean BP levels. This metric enables tailored interventions focused on reducing variability while maintaining target BP levels. In some examples, CoV-BP is represented as a percentage.

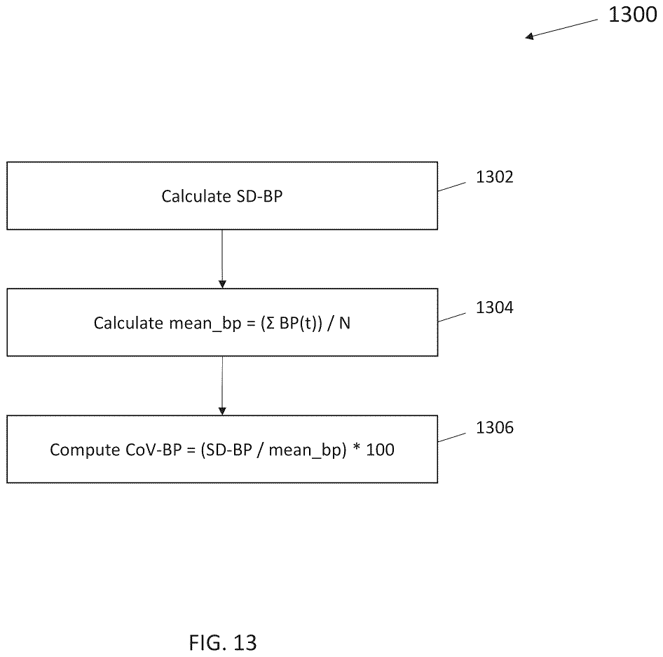

FIG. 13 is a flowchart of a method 1300 for determining CoV-BP in accordance with aspects described herein. In some examples, the metric engine 130 is configured to perform the method 1300, at least in part.

At step 1302, the metric engine 130 calculates SD-BP for the BP data (e.g., using method 1200 of FIG. 12).

At step 1304, the metric engine 130 calculates the mean BP of the BP data (i.e., the mean of BP signal BP(t)). In some examples, the metric engine 130 calculates the mean BP as mean_bp=(ΣBP(t))/N, where N is the total number of readings.

At step 1306, the metric engine 130 determines the value of CoV-BP for the measurement period (e.g. the length of BP(t)). In some examples, the metric engine 130 computes CoV-BP as CoV-BP=(SD-BP/mean_bp)×100.

In some examples, the system 100 is configured to calculate an Average Squared Real Variability (ASRV-BP). ASRV-BP quantifies variability by calculating the average squared difference between consecutive BP readings over the monitoring period. It captures short-term BP fluctuations, providing insight in-to the dynamics of BP control. ASRV-BP highlights the presence of frequent and significant BP swings, which can cause vascular stress and increase the risk of cardiovascular complications. This metric is particularly useful for evaluating the effectiveness of treatments aimed at stabilizing BP, such as slow-release antihypertensive medications or lifestyle interventions. In some examples. ASRV-BP is represented in units of mmHg².

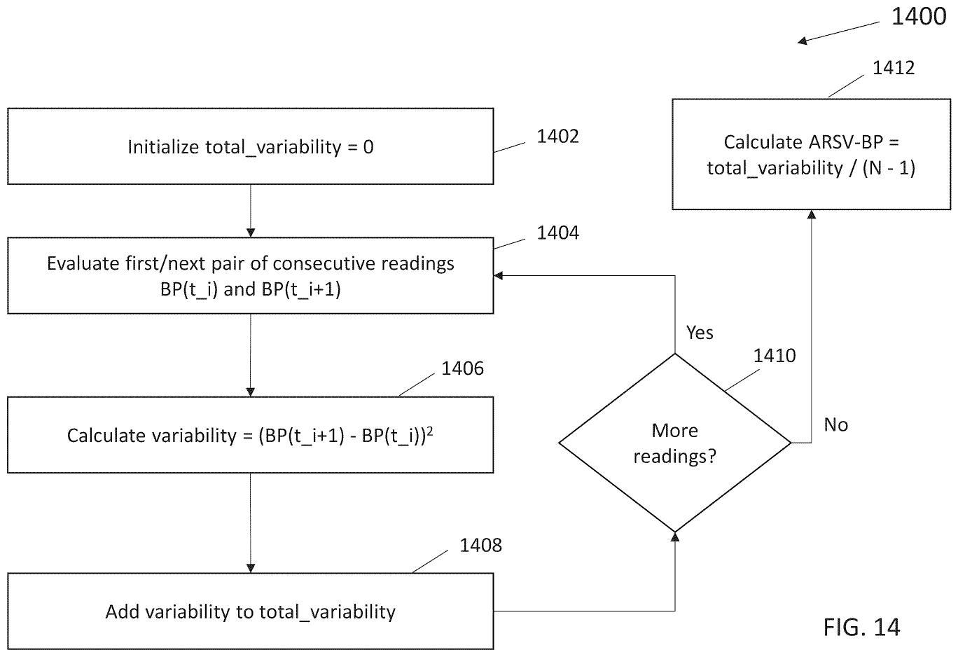

FIG. 14 is a flowchart of a method 1400 for determining ASRV-BP in accordance with aspects described herein. In some examples, the metric engine 130 is configured to perform the method 1400, at least in part.

At step 1402, the metric engine 130 initializes total_variability=0. In some examples, total_variability is initialized each time ASRV-BP is calculated.

At step 1404, the metric engine 130 evaluates a first pair of consecutive readings of the BP data. The consecutive readings are represented as BP(t_i) and BP(t_i+1). In some examples, the consecutive readings correspond to consecutive time intervals dt of the BP signal BP(t).

At step 1406, the metric engine 130 calculates the variability of the first pair of consecutive readings. In some examples, the metric engine 130 calculates variability as variability=(BP(t_(i+1))−BP(t_(i)))².

At step 1408, the metric engine 130 adds the calculated variability to total_variability.

At step 1410, the metric engine 130 determines whether there are more pairs of consecutive readings of the BP data to evaluate. In other words, the metric engine 130 determines whether the complete BP(t) signal has been processed, indicating the end of the ASRV-BP measurement period. If there are no more pairs to evaluate, the method proceeds to step 1412. Otherwise, the method returns to step 1404 and repeats for each pair of consecutive readings.

At step 1414, the metric engine 130 determines the value of ARSV-BP for the measurement period (e.g. the length of BP(t)). In some examples, the metric engine 130 computes ARSV-BP as ARSV-BP=total_variability/(N−1), where N is the total number of readings.

In some examples, the system 100 is configured to calculate a Maximum BP Excursion Duration (MaxBED) metric. MaxBED identifies the longest continuous period during which the user's BP remains outside the target range without returning to normal levels. It measures the single most extended episode of hypertension or hypotension during the monitoring period. Extended periods of uncontrolled BP significantly increase the risk of acute events like stroke, heart attack or rupture of aortic aneurisms. MaxBED helps clinicians pinpoint these high-risk episodes, enabling targeted investigations into potential causes such as missed medications or acute stressors. Addressing these factors can prevent future prolonged BP excursions and reduce immediate health risks. In some examples, MaxBED is represented in units of hours.

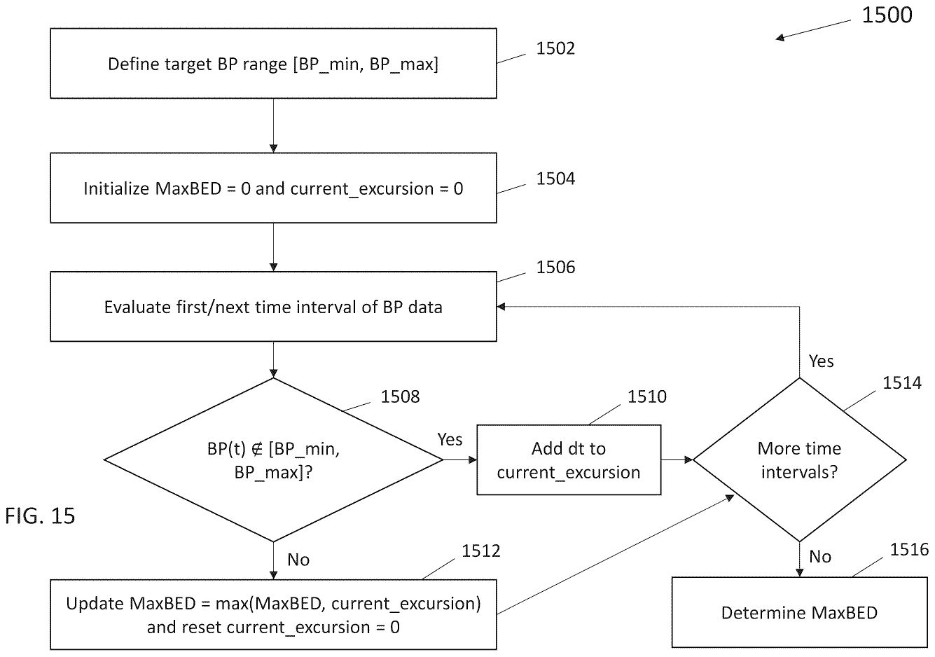

FIG. 15 is a flowchart of a method 1500 for determining MaxBED in accordance with aspects described herein. In some examples, the metric engine 130 is configured to perform the method 1500, at least in part.

At step 1502, the metric engine 130 defines the target BP range [BP_min, BP_max].

At step 1504, the metric engine 130 initializes MaxBED=0 and current_excursion=0. In some examples, MaxBED and current_excursion are both initialized each time MaxBED is calculated.

At step 1506, the metric engine 130 evaluates the first time interval dt of the BP data.

At step 1508, the metric engine 130 determines whether BP(t)∉[BP_min, BP_max] during the first time interval dt. If so, the method proceeds to step 1510. Otherwise, the method proceeds to step 1512.

At step 1510, in response to a determination that BP(t) V [BP_min, BP_max] during the first time interval dt, the metric engine 130 adds dt to current_excursion. In other words, the length of dt is added to current_excursion.

At step 1512, in response to a determination that BP(t) E [BP_min, BP_max] during the first time interval dt, the metric engine 130 updates MaxBED=max(MaxBED, current_excursion) and resets current_excursion=0.

At step 1514, the metric engine 130 determines whether there are more time intervals dt of the BP data to evaluate. In other words, the metric engine 130 determines whether the complete BP(t) signal has been processed, indicating the end of the MaxBED measurement period. If there are no more time intervals dt to evaluate, the method proceeds to step 1516. Otherwise, the method returns to step 1506 and repeats for each time interval dt until the complete BP signal has been processed.

At step 1516, the metric engine 130 determines the value of MaxBED for the measurement period (e.g. the length of BP(t)). In some examples, the metric engine 130 selects the duration of the longest excursion as MaxBED.

In some examples, the system 100 is configured to calculate a Symmetrical Weighting Index (SWI) metric. The SWI assigns weights to BP readings based on their deviation from the target range, applying exponentially increasing weights for larger deviations on both the hypertensive and hypotensive sides. It provides a balanced assessment of risks associated with BP being too high or too low. SWI emphasizes the dangers of extreme BP values in either direction, recognizing that both hypertension and hypotension can have adverse effects. A high SWI alerts clinicians to significant BP fluctuations that may necessitate careful medication titration to avoid overcorrection. This marker supports the development of treatment plans that maintain BP within a safe range, minimizing the risk of adverse events from both high and low BP. In some examples, SWI is represented in arbitrary units.

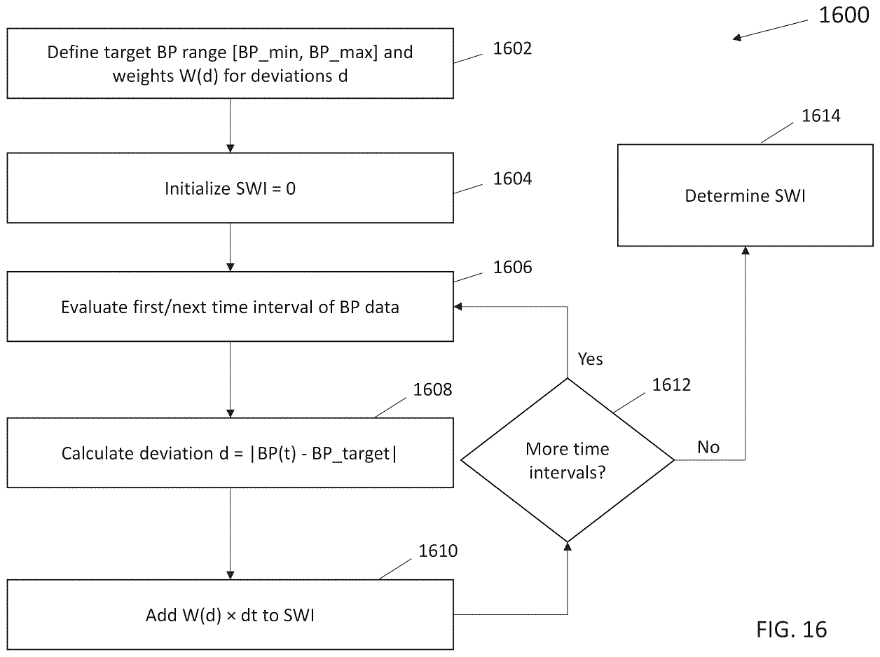

FIG. 16 is a flowchart of a method 1600 for determining SWI in accordance with aspects described herein. In some examples, the metric engine 130 is configured to perform the method 1600, at least in part.

At step 1602, the metric engine 130 defines the target BP range [BP_min, BP_max] and weights W(d) for deviations d. In some examples, W(d) is predefined as a linear or exponential weight function. In some examples, the weights W(d) are individualized for each user (e.g., based on characteristics, pre-existing health conditions, BP related diagnoses, etc.). In some examples, the same weights W(d) are used for each user.

At step 1604, the metric engine 130 initializes SWI=0. In some examples, SWI is initialized each time SWI is calculated.

At step 1606, the metric engine 130 evaluates the first time interval dt of the BP data BP(t).

At step 1608, the metric engine 130 calculates a deviation d for the first time interval dt. The deviation d may be calculated as d=|BP(t)−BP_target|. In some examples, BP_target is the midpoint of [BP_min, BP_max].

At step 1610, the metric engine 130 adds W(d)×dt to SWI.

At step 1612, the metric engine 130 determines whether there are more time intervals dt of the BP data to evaluate. In other words, the metric engine 130 determines whether the complete BP(t) signal has been processed, indicating the end of the SWI measurement period. If there are no more time intervals dt to evaluate, the method proceeds to step 1614. Otherwise, the method returns to step 1606 and repeats for each time interval dt until the complete BP signal has been processed (e.g., by steps 1608 and 1610).

At step 1614, the metric engine 130 determines the value of SWI for the measurement period (e.g., the length of BP(t)). In some examples, the metric engine 130 computes SWI as the sum of weighted deviations d over time.

In some examples, the system 100 is configured to calculate an Acute Hypertensive Event Index with Extreme Non-Linearity (AHEI+) metric. The AHEI+ metric quantifies acute hypertensive events with an emphasis on extreme BP spikes. It applies a highly non-linear weighting function (e.g., exponential or cubed) to systolic BP values exceeding a specified threshold, amplifying the impact of severe BP elevations. AHEI+ is highly sensitive to extreme BP values that may precede cardiovascular accidents like stroke or heart attack. It is valuable for identifying critical events requiring immediate intervention. Severe BP spikes impose acute stress on the vascular system and can lead to life-threatening complications. In some examples, the AHEI+ metric is represented in mmHgⁿ·minutes (e.g., n=3 or 4 for cubic or quartic weighting).

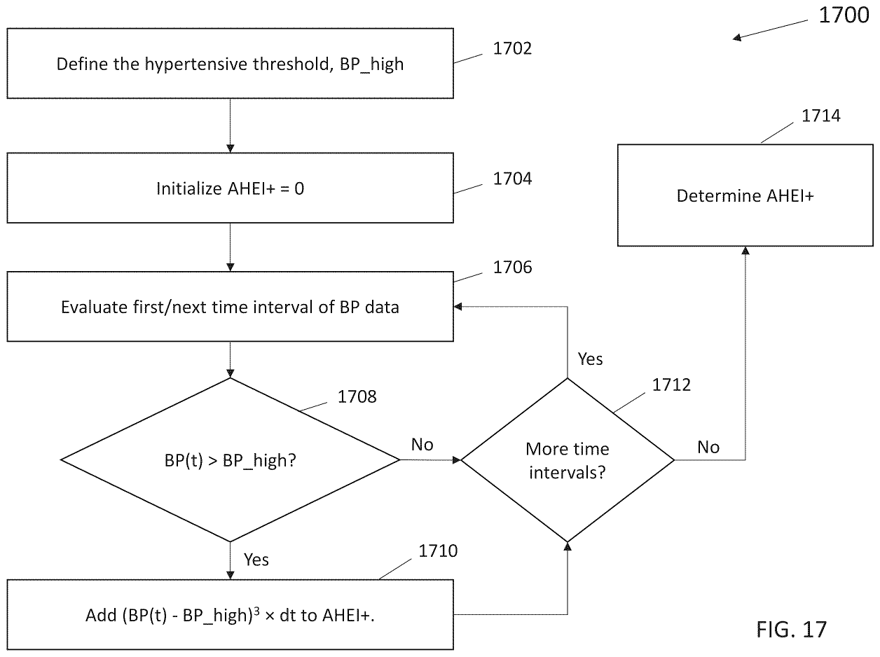

FIG. 17 is a flowchart of a method 1700 for determining AHEI+ in accordance with aspects described herein. In some examples, the metric engine 130 is configured to perform the method 1700, at least in part.

At step 1702, the metric engine 130 defines a hypertensive threshold BP_high. In some examples, BP_high is individualized for each user (e.g., based on characteristics, pre-existing health conditions, BP related diagnoses, etc.). In some examples, the same BP_high is used for each user.

At step 1704, the metric engine 130 initializes AHEI+=0. In some examples, AHEI+ is initialized each time AHEI+ is calculated.

At step 1706, the metric engine 130 evaluates the first time interval dt of the BP data.

At step 1708, the metric engine 130 determines whether BP(t)>BP_high during the first time interval dt. If so, the method proceeds to step 1710. Otherwise, the method proceeds to step 1712.

At step 1710, in response to a determination that BP(t)>BP_high during the first time interval dt, the metric engine 130 adds (BP(t)−BP_high)³×dt to AHEI+.

At step 1712, the metric engine 130 determines whether there are more time intervals dt of the BP data to evaluate. In other words, the metric engine 130 determines whether the complete BP(t) signal has been processed, indicating the end of the AHEI+ measurement period. If there are no more time intervals dt to evaluate, the method proceeds to step 1714. Otherwise, the method returns to step 1706 and repeats for each time interval dt until the complete BP signal has been processed.

At step 1714, the metric engine 130 determines the value of AHEI+ for the measurement period (e.g. the length of BP(t)). In some examples, AHEI+ is expressed in mmHg³·minutes.

In some examples, the system 100 is configured to calculate an Acute Hypertensive Event Index with Dynamic Spike Reinforcement (AHEI++) metric. The AHEI++ metric extends AHEI+ by incorporating the dynamic rate of BP change. Rapid increases in BP, in addition to high absolute values, are given higher weight to capture sudden, dangerous hypertensive surges. Rapid BP surges impose acute stress on vascular integrity and can precipitate catastrophic cardiovascular events. AHEI++ enables detection of dynamic, high-risk BP behavior that may not be apparent from static BP values alone. In some examples, the AHEI++ metric is represented in mmHgⁿ·minutes (e.g., n=3 or 4 for cubic or quartic weighting).

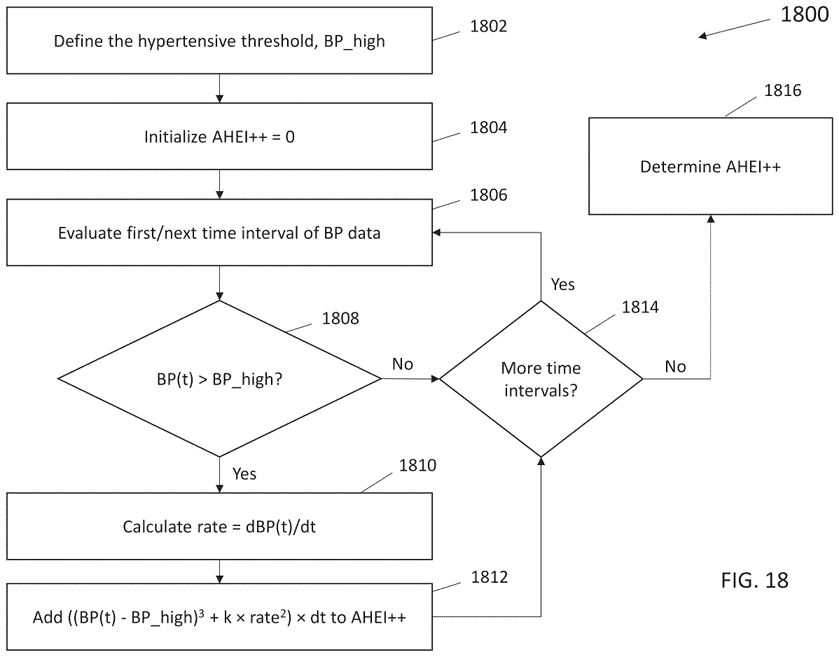

FIG. 18 is a flowchart of a method 1800 for determining AHEI++ in accordance with aspects described herein. In some examples, the metric engine 130 is configured to perform the method 1800, at least in part.

At step 1802, the metric engine 130 defines a hypertensive threshold BP_high. In some examples, BP_high is individualized for each user (e.g., based on characteristics, pre-existing health conditions, BP related diagnoses, etc.). In some examples, the same BP_high is used for each user.

At step 1804, the metric engine 130 initializes AHEI++=0. In some examples, AHEI++ is initialized each time AHEI++ is calculated.

At step 1806, the metric engine 130 evaluates the first time interval dt of the BP data.

At step 1808, the metric engine 130 determines whether BP(t)>BP_high during the first time interval dt. If so, the method proceeds to step 1810. Otherwise, the method proceeds to step 1814.

At step 1810, in response to a determination that BP(t)>BP_high during the first time interval dt, the metric engine 130 calculates a rate of change for the user's BP. In some examples, the rate of change is calculated as rate=dBP(t)/dt.

At step 1812, the metric engine 130 adds ((BP(t)−BP_high)³+k×rate²)×dt to AHEI++. In some examples, k is a scaling factor that enhances the importance (or weight) given to rapid BP increases. For example, setting k=1 means that more importance is given to the amount of BP excess (e.g., over BP_high) than to the rate at which the user's BP is increasing. Likewise, setting k=5 means that more importance is given to the rate at which the user's BP is increasing, irrespectively of the amount of BP excess. By emphasizing BP rate over BP excess, the metric engine 130 can capture cases where the user's BP is only marginally high but rapidly increasing.

At step 1814, the metric engine 130 determines whether there are more time intervals dt of the BP data to evaluate. In other words, the metric engine 130 determines whether the complete BP(t) signal has been processed, indicating the end of the AHEI++ measurement period. If there are no more time intervals dt to evaluate, the method proceeds to step 1816. Otherwise, the method returns to step 1806 and repeats for each time interval dt until the complete BP signal has been processed.

At step 1816, the metric engine 130 determines the value of AHEI++ for the measurement period (e.g. the length of BP(t)). In some examples, AHEI++ is expressed in mmHg³ minutes.

In some examples, the system 100 is configured to calculate a Chronic Hypertensive Exposure Index with Non-linear Time Reinforcement (CHEI+) metric. The CHEI+ metric quantifies prolonged exposure to elevated BP, applying non-linear weighting to the duration of exposure. Long, uninterrupted periods of hypertension are emphasized to reflect their greater impact on cardiovascular health. Extended hypertension episodes are a key driver of long-term cardiovascular and renal damage. CHEI+ identifies patients at heightened risk due to sustained BP elevation, supporting targeted interventions to reduce chronic BP burden. In some examples, the CHEI+ metric is represented in mmHg·hoursⁿ (e.g., n=2 or 3 based on weighting).

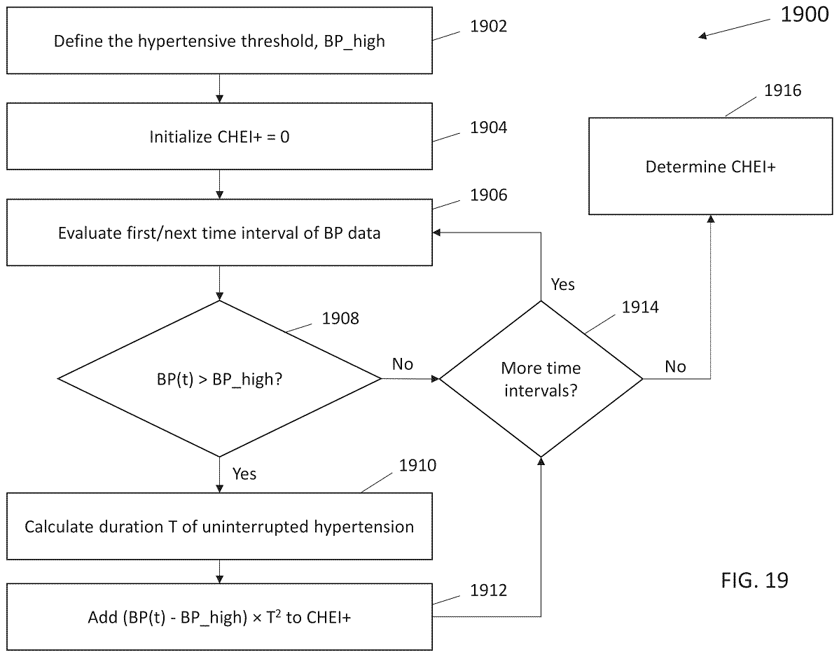

FIG. 19 is a flowchart of a method 1900 for determining CHEI+ in accordance with aspects described herein. In some examples, the metric engine 130 is configured to perform the method 1900, at least in part.

At step 1902, the metric engine 130 defines a hypertensive threshold BP_high. In some examples, BP_high is individualized for each user (e.g., based on characteristics, pre-existing health conditions, BP related diagnoses, etc.). In some examples, the same BP_high is used for each user.

At step 1904, the metric engine 130 initializes CHEI+=0. In some examples, CHEI+ is initialized each time CHEI+ is calculated.

At step 1906, the metric engine 130 evaluates the first time interval dt of the BP data.

At step 1908, the metric engine 130 determines whether BP(t)>BP_high during the first time interval dt. If so, the method proceeds to step 1910. Otherwise, the method proceeds to step 1914.

At step 1910, in response to a determination that BP(t)>BP_high during the first time interval dt, the metric engine 130 calculates a duration T of uninterrupted hypertension (i.e., the amount of time that BP(t)>BP_high).

At step 1912, the metric engine 130 adds (BP(t)−BP_high)×T² to CHEI+.

At step 1914, the metric engine 130 determines whether there are more time intervals dt of the BP data to evaluate. In other words, the metric engine 130 determines whether the complete BP(t) signal has been processed, indicating the end of the CHEI+ measurement period. If there are no more time intervals dt to evaluate, the method proceeds to step 1916. Otherwise, the method returns to step 1906 and repeats for each time interval dt until the complete BP signal has been processed.

At step 1916, the metric engine 130 determines the value of CHEI+ for the measurement period (e.g. the length of BP(t)). In some examples, CHEI+ is expressed in mmHg²-hours.

In some examples, the system 100 is configured to calculate a Combined Acute and Chronic Index (CACI) metric. CACI integrates the acute (AHEI++) and chronic (CHEI+) metrics into a single value, providing a comprehensive assessment of both short-term spikes and prolonged hypertension exposure. CACI offers a balanced view of acute and chronic BP risks, enabling clinicians to address both immediate threats and long-term health deterioration. In some examples, CACI is a dimensionless marker (e.g., normalized sum of AHEI++ and CHEI+).

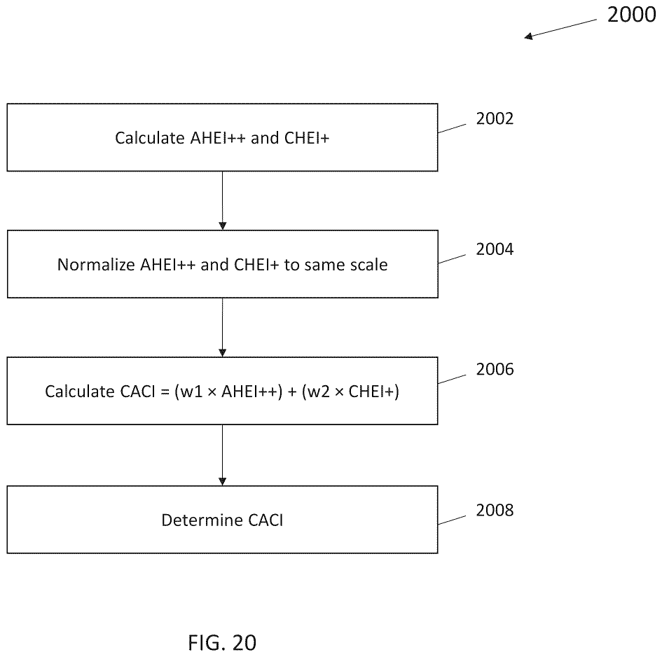

FIG. 20 is a flowchart of a method 2000 for determining CACI in accordance with aspects described herein. In some examples, the metric engine 130 is configured to perform the method 2000, at least in part.

At step 2002, the metric engine 130 calculates AHEI++ and CHEI+ for the BP data (e.g., as described in methods 1800 and 1900, respectively).

At step 2004, the metric engine 130 normalizes AHEI++ and CHEI+ to the same scale. Given that these markers are calculated with different configurations, they may have values with different orders of magnitude (e.g., one may be 10 and the other 1,000). As such, combining the raw values of the markers may cause one marker to overtake the other. The normalization step aims at bringing the two values to a similar scale such that they can be combined properly. For example, AHEI++ values having an order of magnitude of 10 may be divided by 10 and CHEI+ values having an order of magnitude of 1,000 may be divided by 1,000. In this manner, the two normalized markers are oscillating around “1”, and can be visualized in a more convenient way.

At step 2006, the metric engine 130 computes the combined CACI metric using the normalized AHEI++ and CHEI+ markers. In some examples, the metric engine 130 computes CACI as CACI=(w1×AHEI++)+(w2×CHEI+), where w1 and w2 are weighting factors. The weighting factors w1 and w2 may be used to balance the importance (or effect) of each marker in determining CACI. For example, the user, or the healthcare provider, may want to emphasize one marker over the other. As such, the metric engine 130 adapts the weights accordingly.

At step 2008, the metric engine 130 determines the value of CACI for the measurement period (e.g. the length of BP(t)). In some examples, the metric engine 130 expresses CACI as a dimensionless score.

In some examples, the system 100 is configured to provide a graphical representation of the acute (AHEI++) and chronic (CHEI+) metrics in two or three dimensions. In such examples, the representation visually highlights the relationship between short-term spikes and long-term hypertension exposure. Visualization aids in identifying patients with mixed risk profiles and tailoring interventions accordingly. Clinicians can prioritize addressing acute spikes, chronic exposure, or both based on the visual representation. Clustering and automatic classification of subjects based on their patterns in these visual representations can guide clinicians in the diagnosis and management of the condition and can be the input to automatic diagnosis and management machine learning algorithms (e.g., via the AI engine 132). In some examples, the graphical representation includes a first axis for AHEI++ (e.g., in units of mmHg³·minutes) and a second axis for CHEI+ (e.g., in units of mmHg·hours²). The graphical representation may include an optional third axis for temporal trends.

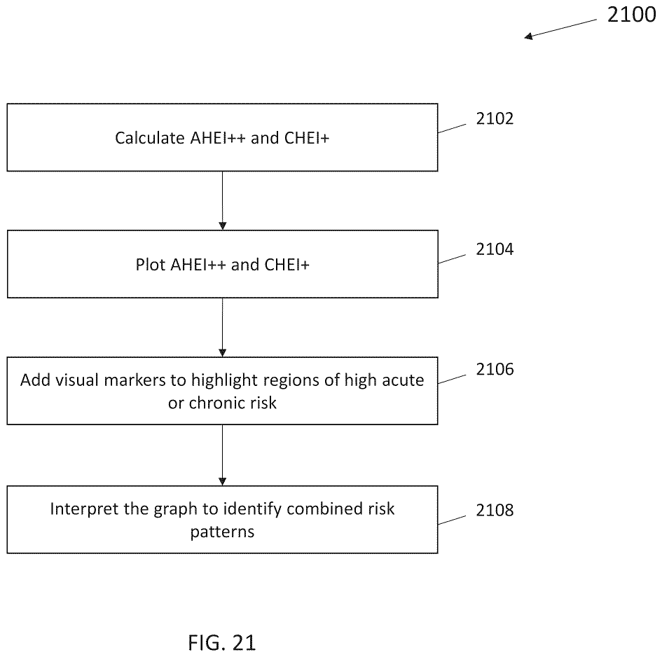

FIG. 21 is a flowchart of a method 2100 for generating a graphical representation of AHEI++ and CHEI+ in accordance with aspects described herein. In some examples, the metric engine 130 is configured to perform the method 2100, at least in part.

At step 2102, the metric engine 130 calculates AHEI++ and CHEI+ for the BP data (e.g., as described in methods 1800 and 1900, respectively).

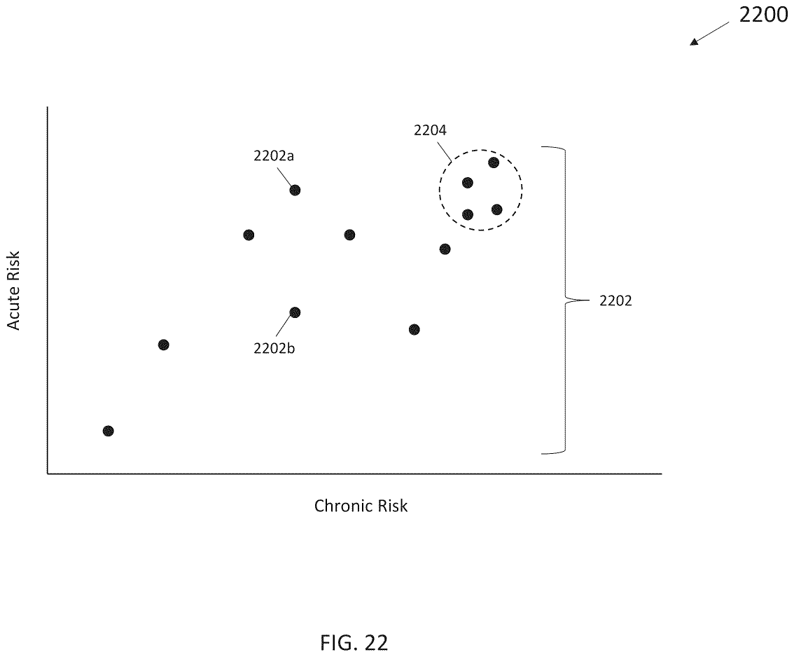

At step 2104, the metric engine 130 plots AHEI++ and CHEI+. In some examples, the metric engine 130 plots AHEI++ and CHEI+ on a 2D plane. In some examples, the metric engine 130 plots AHEI++ and CHEI+ in 3D space with time as the third axis. FIG. 22 illustrates an example plot 2200 in accordance with aspects described herein. As shown, the x-axis represents chronic risk (i.e., CHEI+) and the y-axis represents acute risk (i.e., AHEI++). The plot 2200 includes a plurality of points 2202. In some examples, each point 2200 represents an AHEI++ and CHEI+ pair of a particular user. For example, each point represents AHEI++ and CHEI+ values corresponding to BP data collected over the same time period. In some examples, each point represents the most recent AHEI++ and CHEI+ values for a user. In some examples, the plot 2200 enables all users to be viewed together. In some examples, groups of users are clustered according to where they are in the graphic. For example, cluster 2204 indicates a group of users having both high acute and high chronic risks. The plot 2200 may include multiple points for the same user. In one example, a first point 2202a corresponds to AHEI++ and CHEI+ values for user 110 at time T1 and a second point 2202b corresponds to AHEI++ and CHEI+ values for user 110 at a later time T2. As such, the trajectory of the points 2202a, 2202b may be used to measure the efficacy of an intervention plan (or treatment). For example, if a user was in the upper right corner at the beginning of the intervention, and after one month the intervention managed to move the user lower, it can be determined that the intervention was successful in decreasing the user's acute risk (i.e., reducing AHEI++). In some examples, this determination may be used to adjust the intervention plan to emphasize a decrease in chronic risk. Likewise, if a user is not moving at all during the intervention (i.e., between times T1 and T2), it may be determined that the intervention is not efficient and needs to be adapted.

At step 2106, the metric engine 130 adds visual markers to highlight regions of high acute or chronic risk. In some examples, the visual markers include grids, quadrants, or regions that correspond to different classifications of risk. In some examples, the metric engine 130 adds visual markers that map the trajectory of individual users over time providing a visual representation of the natural change in health status of the user, or the change of health status during the implementation of a given intervention (providing an estimate of the efficiency of the recommended intervention). The visual markers can also map the trajectory of a group of users that share similar characteristics or that have been exposed to similar recommended interventions. The grouping of users can be done based on at least one of the parameters disclosed herein, including but not limited to physiological characteristics, sociodemographic characteristics, cardiovascular characteristics, engagement patterns or cohorts generated during A/B testing of interventions. Metrics on individual or group health status can be derived from these trajectories (e.g., based on the length of the trajectory, its direction, its velocity, its acceleration, or any other characterization of a trajectory in different scales in time). Similar metrics can be derived to calculate the efficiency of an intervention on an individual user or a group of users (e.g., based on the same parameters or characteristics described before, or possibly adding information on the strength, duration, type of intervention, sequence of interventions, or grouping of interventions).

At step 2108, the metric engine 130 (or the user, physician, or medical practitioner) interprets the graph to identify combined risk patterns. In some examples, the combined risk patterns vary depending on the context of the user. For subjects with points in the high chronic risk regions of the plot 2200, BP control is important for long-term (i.e., chronic) outcomes of heart failure, arrhythmias, coronary artery disease, stroke, dementia, vascular diseases including aneurysms, kidney diseases/failure, diabetes, vision loss, erectile dysfunction, and many others. For these subjects, reductions in chronic risk are prioritized over reductions in acute risk. Such subjects may include hypertensive subjects, particularly those less than 65 years old, subjects with high HL or CBPL (irrespective of HTN diagnosis), subjects with low TTR (irrespective of HTN diagnosis), subjects with a family history of the above diseases, and subjects with a genetic predisposition of the above diseases. As such, the ideal risk pattern for a young hypertensive subject may be for the subject's point(s) to navigate in the left side of the plot 2200. This pattern would indicate that the subject's chronic risk level has been reduced. Even if the subject experiences increased BP because they have an active life, the potential harm is reduced due to the reduction in chronic risk. Similarly, for subjects with points in the high acute risk regions of the plot 2200, BP control is important for short-term (i.e., acute) outcomes to prevent recurrent hospitalization or recurrent event. For these subjects, reductions in acute risk are prioritized over reductions in chronic risk. Such subjects may include subjects with congestive heart failure, a recent stroke, an aortic aneurysm, and subjects with Eclampsia, pre-Eclampsia, or pregnancy induced HTN. As such, for an a older patient with an aortic aneurysm, the ideal risk pattern would be any pattern that indicates a reduction in acute risk (i.e., because increases in acute risk could be fatal). Therefore, the ideal risk pattern for such a subject's point(s) is to navigate in the lower half of the plot 2200.

In some examples, the system 100 is configured to calculate a Consecutive Hypertensive Hours Index (CHHI). The CHHI quantifies the percentage of days over a one-year period where the number of consecutive hours above a defined BP threshold exceeds a specified value. This metric captures the recurrence of extended hypertensive episodes within a daily timeframe. Frequent daily periods of sustained hypertension significantly elevate the risk of acute cardiovascular events and end-organ damage. CHHI provides insights into recurring hypertensive patterns that may require targeted therapeutic adjustments or lifestyle interventions to mitigate long-term health risks. In some examples, CHHI is represented as a percentage of days (e.g., 15% of days in a year).

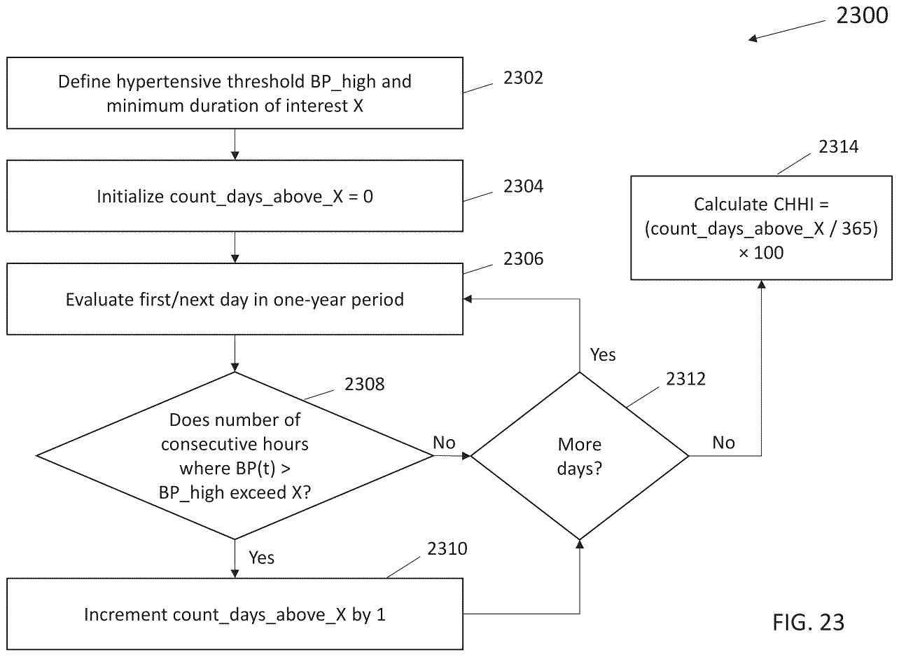

FIG. 23 is a flowchart of a method 2300 for determining CHHI in accordance with aspects described herein. In some examples, the metric engine 130 is configured to perform the method 2300, at least in part.

At step 2302, the metric engine 130 defines a hypertensive threshold BP_high and a minimum duration of interest X. In some examples, BP_high and/or duration X is individualized for each user (e.g., based on characteristics, pre-existing health conditions, BP related diagnoses, etc.). In some examples, the same BP_high and/or duration X is used for each user.

At step 2304, the metric engine 130 initializes count_days_above_X=0. In some examples, count_days_above_X is initialized each time CHHI is calculated.

At step 2306, the metric engine 130 evaluates the first day in a one-year measurement period.

At step 2308, the metric engine 130 determines whether the number of consecutive hours where BP(t)>BP_high during the first day exceeds duration X. If so, the method proceeds to step 2310. Otherwise, the method proceeds to step 2312.

At step 2310, in response to a determination that the number of consecutive hours where BP(t)>BP_high during the first day exceeds duration X, the metric engine 130 increments count_days_above_X by 1. Then the method proceeds to step 2312.

At step 2312, the metric engine 130 determines whether there are more days in the one-year measurement period to evaluate. If not, the method proceeds to step 2314. Otherwise, the method returns to step 2306 and repeats for each day until the complete one-year measurement period has been completed.

At step 2314, the metric engine 130 determines the value of CHHI for the one-year measurement period. In some examples, the metric engine 130 calculates CHHI as CHHI=(count_days_above_X/365)×100.

In some examples, the system 100 is configured to calculate a Continuous Hypertensive Exposure Percentage (CHEP). The CHEP quantifies the percentage of time over a one-year period during which the user's BP remained continuously above a defined hypertensive threshold without interruption. This metric emphasizes prolonged unbroken hypertensive exposure. Prolonged, uninterrupted hypertensive episodes impose severe stress on the cardiovascular system, leading to progressive damage to organs and increased risk of chronic conditions. CHEP identifies patients at risk due to sustained BP elevation, allowing for precise and proactive intervention strategies. In some examples, the CHEP is a percentage of time.

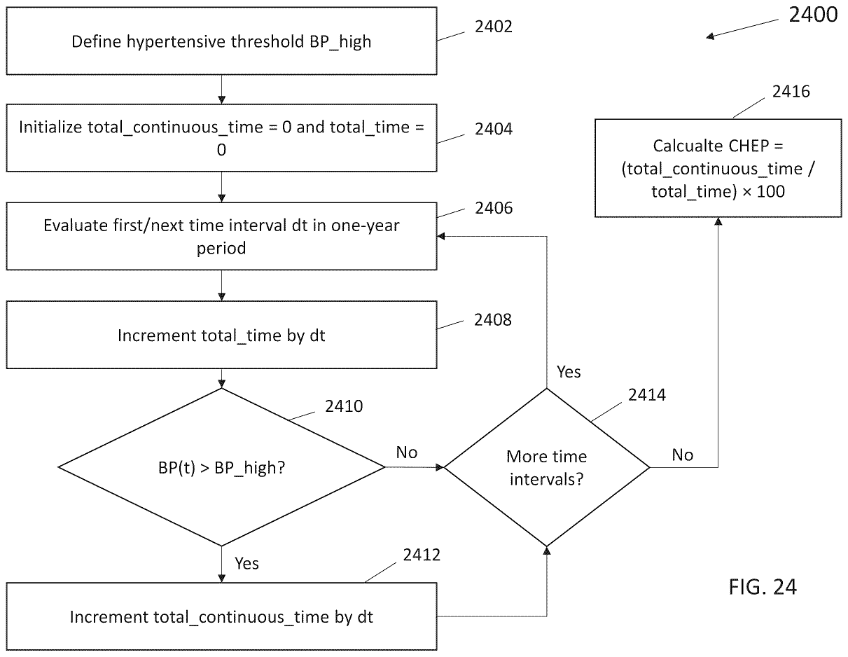

FIG. 24 is a flowchart of a method 2400 for determining CHEP in accordance with aspects described herein. In some examples, the metric engine 130 is configured to perform the method 2400, at least in part.

At step 2402, the metric engine 130 defines a hypertensive threshold BP_high. In some examples, BP_high is individualized for each user (e.g., based on characteristics, pre-existing health conditions, BP related diagnoses, etc.). In some examples, the same BP_high is used for each user.

At step 2404, the metric engine 130 initializes total_continuous_time=0 and total_time=0. In some examples, total_continuous_time and total_time are both initialized each time CHEP is calculated.

At step 2406, the metric engine 130 evaluates the first time interval dt in a one-year measurement period.

At step 2408, the metric engine 130 increments total_time by dt. In other words, the length of the first time interval dt is added to total_time

At step 2410, the metric engine 130 determines whether BP(t)>BP_high during the first time interval dt. If so, the method proceeds to step 2412. Otherwise, the method proceeds to step 2414.

At step 2412, in response to a determination that BP(t)>BP_high during the first time interval dt, the metric engine 130 increments total_continuous_time by dt. In other words, the length of the first time interval dt is added to total_continuous_time.

At step 2414, the metric engine 130 determines whether there are more time intervals dt of the BP data to evaluate. In other words, the metric engine 130 determines whether the complete BP(t) signal has been processed, indicating the end of the CHEP measurement period. If there are no more time intervals dt to evaluate, the method proceeds to step 2416. Otherwise, the method returns to step 2406 and repeats for each time interval dt until the complete BP signal has been processed.

At step 2416, the metric engine 130 determines the value of CHEP for the one-year measurement period. In some examples, the metric engine 130 calculates CHEP as CHEP=(total_continuous_time/total_time)×100.

In some examples, the system 100 is configured to calculate a Persistent Hypertension Days Index (PHDI). The PHDI quantifies the number of days in a year where a user's BP never drops below a defined threshold for the entire 24-hour period. This metric captures the presence of continuous hypertension on a daily basis, highlighting sustained high BP that may indicate significant cardiovascular risk. Persistent daily hypertension without any return to normal BP levels places a severe, cumulative burden on the cardiovascular system. This prolonged exposure can accelerate organ damage, increase the risk of heart failure, and contribute to long-term morbidity. PHDI helps identify patients with chronic, uncontrolled hypertension and guides healthcare providers in implementing aggressive management strategies to reduce the sustained BP burden. In some examples, PHDI is represented as a number of days per year (e.g., 10) or a percentage of days per year (e.g., 10%).

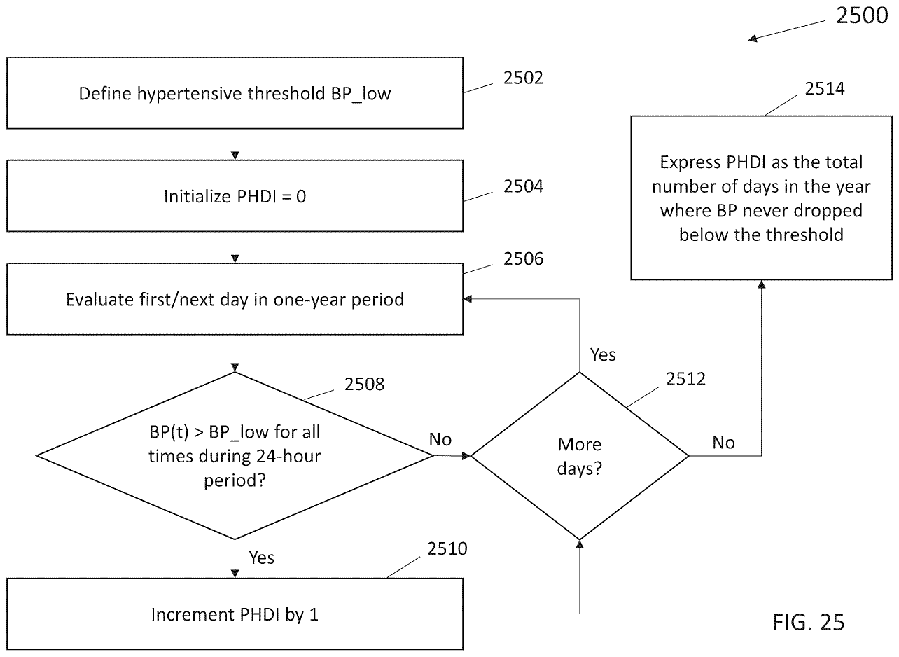

FIG. 25 is a flowchart of a method 2500 for determining PHDI in accordance with aspects described herein. In some examples, the metric engine 130 is configured to perform the method 2500, at least in part.

At step 2502, the metric engine 130 defines a hypertensive threshold BP_low. In some examples, BP_low is individualized for each user (e.g., based on characteristics, pre-existing health conditions, BP related diagnoses, etc.). In some examples, the same BP_low is used for each user.

At step 2504, the metric engine 130 initializes PHDI=0. In some examples, PHDI is initialized each time PHDI is calculated.

At step 2506, the metric engine 130 evaluates the first day in a one-year measurement period.

At step 2508, the metric engine 130 determines whether BP(t)>BP_low for all times during the 24-hour period of the first day. If so, the method proceeds to step 2510. Otherwise, the method proceeds to step 2512.

At step 2510, in response to a determination that BP(t)>BP_low for all times during the 24-hour period of the first day, the metric engine 130 increments PHDI by 1. Then the method proceeds to step 2512.

At step 2512, the metric engine 130 determines whether there are more days in the one-year measurement period to evaluate. If not, the method proceeds to step 2514. Otherwise, the method returns to step 2506 and repeats for each day until the complete one-year measurement period has been completed.

At step 2514, the metric engine 130 determines the value of PHDI for the one-year measurement period. In some examples, the metric engine 130 expresses PHDI as the total number of days in the year where BP never dropped below the threshold (i.e., BP_low).

In some examples, the system 100 is configured to calculate a Rate of BP Normalization (RBPN). RBPN measures the average time it takes for the user's BP to return to the target range after deviating from it. It assesses the body's ability to recover from episodes of hypertension or hypotension. A slower RBPN may indicate impaired vascular responsiveness or suboptimal treatment efficacy. Clinicians can use RBPN to evaluate the need for faster-acting medications or interventions that enhance the body's regulatory mechanisms. Improving the rate at which BP normalizes reduces the duration of exposure to harmful BP levels, thereby decreasing cardiovascular risk. In some examples, RBPN is represented in units of time (e.g., seconds, minutes, etc.).

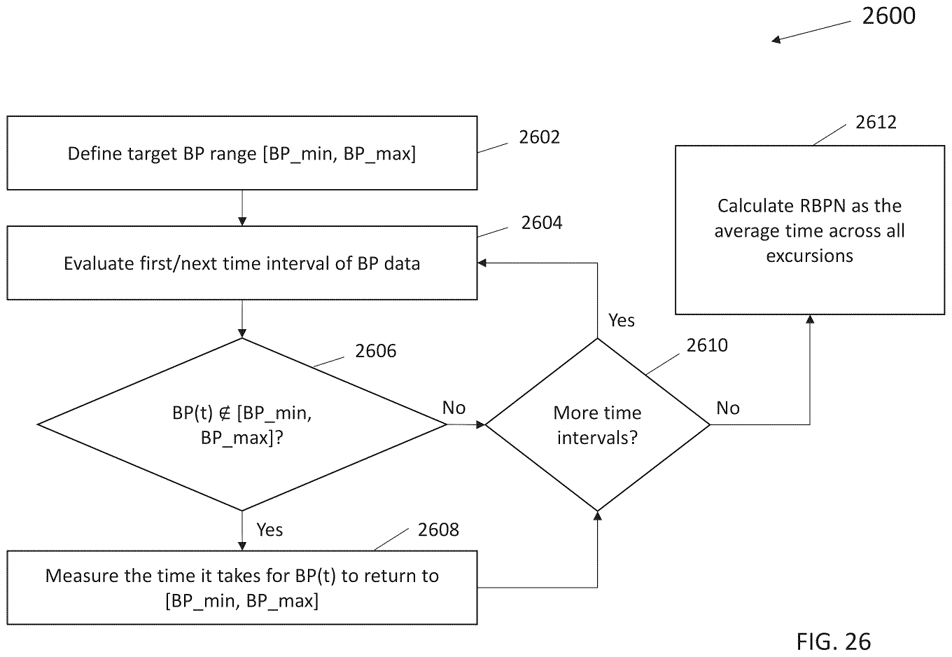

FIG. 26 is a flowchart of a method 2600 for determining RBPN in accordance with aspects described herein. In some examples, the metric engine 130 is configured to perform the method 2600, at least in part.

At step 2602, the metric engine 130 defines the target BP range [BP_min, BP_max].

At step 2604, the metric engine 130 evaluates the first time interval dt of the BP data.

At step 2606, the metric engine 130 determines whether BP(t) V [BP_min, BP_max] during the first time interval dt. If so, the method proceeds to step 2608. Otherwise, the method proceeds to step 2610.

At step 2608, in response to a determination that BP(t) V [BP_min, BP_max] during the first time interval dt, the metric engine 130 measure the time it takes for BP(t) to return to [BP_min, BP_max].

At step 2610, the metric engine 130 determines whether there are more time intervals dt of the BP data to evaluate. In other words, the metric engine 130 determines whether the complete BP(t) signal has been processed, indicating the end of the RBPN measurement period. If there are no more time intervals dt to evaluate, the method proceeds to step 2612. Otherwise, the method returns to step 2604 and repeats for each time interval dt until the complete BP signal has been processed.

At step 2612, the metric engine 130 determines the value of RBPN for the measurement period (e.g. the length of BP(t)). In some examples, the metric engine 130 calculates RBPN as the average time across all excursions (i.e., the average of the measured times from step 2608).

In some examples, the system 100 is configured to calculate a Cumulative Exposure to Extreme BP (CEEB). CEEB quantifies the total time and magnitude that BP readings exceed critical thresholds significantly higher or lower than the standard target range (e.g., systolic BP>180 mmHg or <80 mmHg). CEEB focuses on the exposure to potentially life-threatening BP levels. Exposure to extreme BP levels poses immediate risks such as hypertensive crises or severe hypotension leading to shock. A high CEEB value signals the need for urgent medical intervention. Clinicians can implement emergency action plans, educate patients on recognizing warning signs, and consider prescribing rescue medications. Monitoring CEEB helps prevent acute events by ensuring timely responses to dangerous BP excursions. In some examples, CEEB is represented in mmHg·hours.

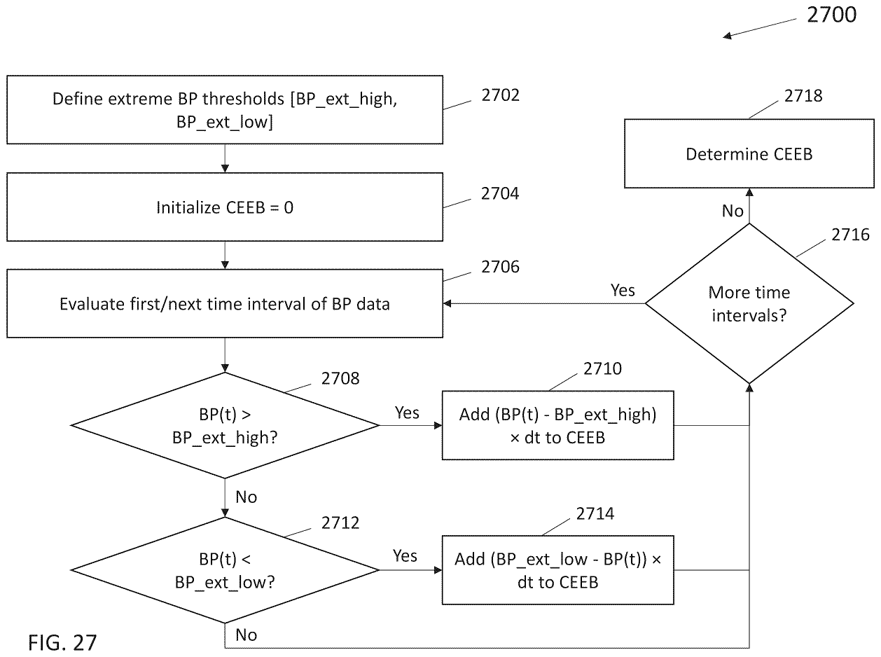

FIG. 27 is a flowchart of a method 2700 for determining CEEB in accordance with aspects described herein. In some examples, the metric engine 130 is configured to perform the method 2700, at least in part.

At step 2702, the metric engine 130 defines extreme BP thresholds [BP_ext_high, BP_ext_min]. In some examples, the extreme BP thresholds correspond to BP values in mmHg (e.g., BP_ext_high=180, BP_ext_low=80).

At step 2704, the metric engine 130 initializes CEEB=0. In some examples, CEEB is initialized each time CEEB is calculated.

At step 2706, the metric engine 130 evaluates the first time interval dt of the BP data.

At step 2708, the metric engine 130 determines whether BP(t)>BP_ext_high during the first time interval dt. If so, the method proceeds to step 2710. Otherwise, the method proceeds to step 2712.

At step 2710, in response to a determination that BP(t)>BP_ext_high during the first time interval dt, the metric engine 130 adds (BP(t)−BP_ext_high)×dt to CEEB. The method then proceeds to step 2716.

At step 2712, the metric engine 130 determines whether BP(t)<BP_ext_low during the first time interval dt. If so, the method proceeds to step 2714. Otherwise, the method proceeds to step 2716.

At step 2714, in response to a determination that BP(t)<BP_ext_low during the first time interval dt, the metric engine 130 adds (BP_ext_low−BP(t))×dt to CEEB. The method then proceeds to step 2716.

At step 2716, the metric engine 130 determines whether there are more time intervals dt of the BP data to evaluate. In other words, the metric engine 130 determines whether the complete BP(t) signal has been processed, indicating the end of the CEEB measurement period. If there are no more time intervals dt to evaluate, the method proceeds to step 2718. Otherwise, the method returns to step 2706 and repeats for each time interval dt until the complete BP signal has been processed.

At step 2718, the metric engine 130 determines the value of CEEB for the measurement period (e.g. the length of BP(t)). In some examples, the metric engine 130 expresses CEEB in mmHg·hours.

In some examples, the system 100 is configured to calculate a Threshold-Based Hypertensive Exposure Vector (THEV). The THEV quantifies the proportion of time an individual's BP exceeds specified thresholds over a defined monitoring period. In some examples, the thresholds include clinically significant values such as BP>130 mmHg, BP>140 mmHg, BP>160 mmHg, and BP>180 mmHg. In such examples, the marker generates a vector where each element corresponds to the percentage of time BP surpasses each threshold. THEV provides a granular understanding of BP control, capturing not just the presence of hypertension but its severity distribution over time. For example, while a patient may spend 20% of their time above 140 mmHg, the additional 5% above 160 mmHg might indicate escalating cardiovascular risk. This marker supports clinicians in tailoring interventions to address both moderate and severe hypertension episodes, reducing long-term damage. In some examples, the THEV is expressed as percentages for each threshold (e.g., % of time BP>130 mmHg, % of time BP>140 mmHg, etc.).

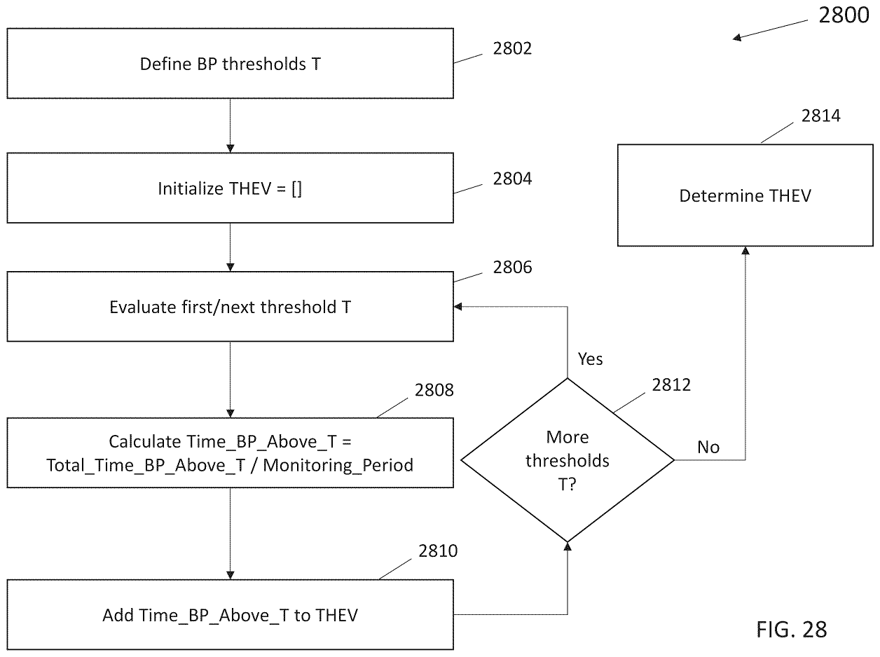

FIG. 28 is a flowchart of a method 2800 for determining a THEV in accordance with aspects described herein. In some examples, the metric engine 130 is configured to perform the method 2800, at least in part.

At step 2802, the metric engine 130 defines two or more BP thresholds T. In some examples, the T thresholds are individualized for each user (e.g., based on characteristics, pre-existing health conditions, BP related diagnoses, etc.). In some examples, the same T thresholds are used for each user. In some examples, the T thresholds correspond to BP values in mmHg (e.g., [130, 140, 160, 180] mmHg).

At step 2804, the metric engine 130 initializes an empty vector THEV=[ ]. In some examples, THEV is initialized each time a THEV is calculated.

At step 2806, the metric engine 130 evaluates the BP data with respect to the first T threshold.

At step 2808, the metric engine 130 calculates the time that the BP data (i.e., BP signal BP(t)) is above the first T threshold. In some examples, the metric engine 130 performs the calculation Time_BP_Above_T=Total_Time_BP_Above_T/Monitoring_Period, where Total_Time_BP_Above_T is the amount of time BP(t) is above the first T threshold and Monitoring_Period is the length of the total measurement period (e.g., the length or duration of BP(t)).

At step 2810, the metric engine 130 adds Time_BP_Above_T to THEV. In some examples, the Time_BP_Above_T value is added to THEV as a percentage.

At step 2812, the metric engine 130 determines whether there are more T thresholds to evaluate. In other words, the metric engine 130 determines whether BP(t) signal has been evaluated with respect to each T threshold, indicating the end of the THEV measurement period. If there are no more T thresholds to evaluate, the method proceeds to step 2814. Otherwise, the method returns to step 2806 and repeats for each T threshold.

At step 2814, the metric engine 130 determines the values of THEV for the measurement period (e.g., the length of BP(t)). In some examples, the metric engine 130 outputs the THEV as a vector of percentages (e.g., [%>130, %>140, %>160, %>180]).

In some examples, the system 100 is configured to generate a Threshold-Based Hypertensive Exposure Curve (THEC). THEC visualizes the relationship between BP thresholds and the proportion of time spent above them over a defined period. In some examples, the x-axis represents thresholds (e.g., BP>130, BP>140, BP>160, BP>180), and the y-axis represents the percentage of time above each threshold. The resulting curve may resemble a sigmoid, highlighting transitions in hypertensive exposure severity. THEC offers a visual tool for assessing hypertension severity, helping healthcare providers quickly identify whether a patient has sporadic or sustained severe hypertension. Clinicians can use this marker to prioritize interventions for thresholds that dominate the patient's exposure curve and evaluate treatment efficacy visually.

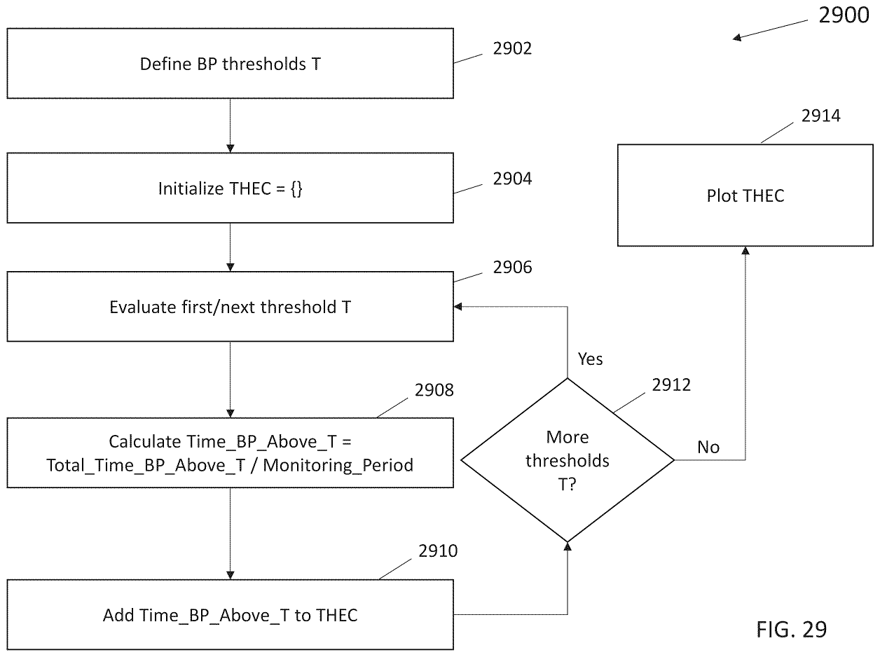

FIG. 29 is a flowchart of a method 2900 for determining a THEC in accordance with aspects described herein. In some examples, the metric engine 130 is configured to perform the method 2900, at least in part.

At step 2902, the metric engine 130 defines two or more BP thresholds T. In some examples, the T thresholds are individualized for each user (e.g., based on characteristics, pre-existing health conditions, BP related diagnoses, etc.). In some examples, the same T thresholds are used for each user. In some examples, the T thresholds correspond to BP values in mmHg (e.g., [130, 140, 160, 180] mmHg).

At step 2904, the metric engine 130 initializes an empty dictionary THEC={ }. In some examples, THEC is initialized each time a THEC is calculated.

At step 2906, the metric engine 130 evaluates the BP data with respect to the first T threshold.

At step 2908, the metric engine 130 calculates the time that the BP data (i.e., BP signal BP(t)) is above the first T threshold. In some examples, the metric engine 130 performs the calculation Time_BP_Above_T=Total_Time_BP_Above_T/Monitoring_Period, where Total_Time_BP_Above_T is the amount of time BP(t) is above the first T threshold and Monitoring_Period is the length of the total measurement period (e.g., the length or duration of BP(t)).

At step 2910, the metric engine 130 adds Time_BP_Above_T to THEC. In some examples, the Time_BP_Above_T value is added to THEC as a percentage.

At step 2912, the metric engine 130 determines whether there are more T thresholds to evaluate. In other words, the metric engine 130 determines whether BP(t) signal has been evaluated with respect to each T threshold, indicating the end of the THEC measurement period. If there are no more T thresholds to evaluate, the method proceeds to step 2914. Otherwise, the method returns to step 2906 and repeats for each T threshold.

At step 2914, the metric engine 130 plots the THEC. In some examples, the metric engine 130 plots the THEC on a graph where the x-axis represents the BP thresholds T and the y-axis represents percentages.

In some examples, the system 100 is configured to calculate a Combined Systolic and Diastolic Hypertensive Marker (CSDHM). The CSDHM quantifies BP control by incorporating separate thresholds for SBP and DBP over a defined period. CSDHM captures both systolic and diastolic hypertension patterns, providing a more comprehensive risk assessment. Elevated SBP and DBP are independent predictors of cardiovascular events, and understanding their interplay allows clinicians to better stratify risk and target specific treatment strategies. In some examples, the CSDHM is represented as a combined vector (e.g., % time SBP>140, % time DBP>90). In some examples, the CSDHM is represented as a two-dimensional visual representation with the x-axis for DBP thresholds and the y-axis for SBP thresholds.

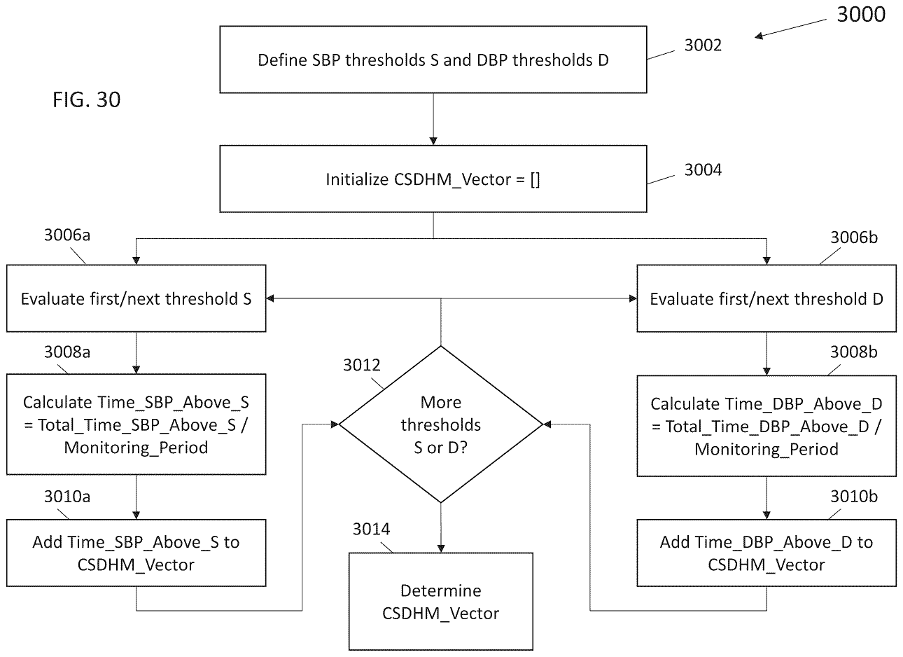

FIG. 30 is a flowchart of a method 3000 for determining CSDHM in accordance with aspects described herein. In some examples, the metric engine 130 is configured to perform the method 3000, at least in part.

At step 3002, the metric engine 130 defines two or more SBP thresholds S and two or more DBP thresholds D. In some examples, the S and/or D thresholds are individualized for each user (e.g., based on characteristics, pre-existing health conditions, BP related diagnoses, etc.). In some examples, the same S and/or D thresholds are used for each user. In some examples, the SBP thresholds S correspond to SBP values in mmHg (e.g., [130, 140, 160, 180] mmHg). Likewise, the DBP thresholds D may correspond to DBP values in mmHg (e.g., [80, 90, 100, 110] mmHg).

At step 3004, the metric engine 130 initializes an empty vector CSDHM_Vector=[ ]. In some examples, CSDHM_Vector is initialized each time CSDHM is calculated. In some examples, the metric engine 130 initializes an empty grid CSDHM_Grid=[ ].

At step 3006a, the metric engine 130 evaluates SBP data with respect to a first S threshold. Likewise, at step 2006b, the metric engine 130 evaluates DBP data with respect to a first D threshold.

At step 3008a, the metric engine 130 calculates the time that the SBP data (i.e., SBP signal SBP(t)) is above the first S threshold. In some examples, the metric engine 130 performs the calculation Time_SBP_Above_S=Total_Time_SBP_Above_S/Monitoring_Period, where Total_Time_SBP_Above_S is the amount of time SBP(t) is above the first S threshold and Monitoring_Period is the length of the total measurement period (e.g., the length or duration of SBP(t)). Likewise, at step 3008b, the metric engine 130 calculates the time that the DBP data (i.e., DBP signal DBP(t)) is above the first D threshold. In some examples, the metric engine 130 performs the calculation Time_DBP_Above_D=Total_Time_DBP_Above_D/Monitoring_Period, where Total_Time_DBP_Above_D is the amount of time DBP(t) is above the first D threshold and Monitoring_Period is the length of the total measurement period (e.g., the length or duration of DBP(t)). In some examples, the value of Monitoring_Period is the same for both the SBP and DBP variations.

At step 3010a, the metric engine 130 adds Time_SBP_Above_S to CSDHM_Vector. In some examples, the Time_SBP_Above_S value is added to CSDHM_Vector as a percentage. Likewise, at step 3010b, the metric engine 130 adds Time_DBP_Above_D to CSDHM_Vector. In some examples, the Time_DBP_Above_D value is added to CSDHM_Vector as a percentage. In some examples, the corresponding Time_SBP_Above_S and Time_DBP_Above_D are added to CSDHM_Grid.

At step 3012, the metric engine 130 determines whether there are more S or D thresholds to evaluate. In other words, the metric engine 130 determines whether the SBP(t) and DBP(t) signals have been evaluated with respect to each of the S or D thresholds, respectively, indicating the end of the CSDHM measurement period. If there are no more S or D thresholds to evaluate, the method proceeds to step 3014. Otherwise, the method returns to steps 3006a, 3006b and repeats for each S or D threshold.

At step 3014, the metric engine 130 determines the values of CSDHM for the measurement period (e.g., the length of SBP(t) and/or DBT(t)). In some examples, the metric engine 130 outputs a combined vector (e.g., [% time SBP>140, % time DBP>90]). In some examples, the metric engine 130 outputs a visual representation. For example, the metric engine 130 may generate a CSDHM plot with an x-axis as DBP thresholds and a y-axis as SBP thresholds showing the combined exposure.

The additional metrics and markers described above expand on the concept of integrating time and magnitude in BP monitoring, offering various approaches to quantify BP control comprehensively. By utilizing such metrics and markers, healthcare providers can gain deeper insights into a patient's cardiovascular risk profile and tailor interventions more effectively.

Integrating time and magnitude in BP monitoring through the use of advanced therapeutic markers offers transformative opportunities for diagnosing, managing, and predicting cardiovascular risk. These markers enable a more nuanced understanding of a patient's BP control over time, accounting for acute and chronic deviations from healthy ranges. By combining these markers into indices or visual representations, healthcare providers can derive actionable insights to tailor short-term interventions and long-term management strategies. Such resources have the potential to revolutionize hypertension care, ultimately reducing the global burden of cardiovascular diseases.

The ability to combine multiple therapeutic markers into a single index represents a significant advance in the field of hypertension management. A composite index can integrate various aspects of BP control-such as acute spikes, chronic elevation, and variability—to provide a comprehensive assessment of cardiovascular risk. Weighting individual markers based on their therapeutic relevance allows the index to be customized for specific endpoints, such as preventing stroke, minimizing kidney damage, or reducing overall cardiovascular mortality. For example, a risk score could prioritize short-term acute events for patients at immediate risk while emphasizing chronic exposure for those with a history of mild hypertension. Tracking the evolution of this composite index over time enables both clinicians and patients to monitor the effectiveness of interventions. For instance, the index can show how a medication regimen or lifestyle change reduces BP excursions or improves TTR, providing tangible evidence of progress and motivation for continued adherence.

The creation of visual representations of combined markers provides an intuitive way to assess cardiovascular risk and therapeutic progress. In a bi-dimensional or multi-dimensional space, each axis could represent a specific marker or a combination thereof. For example, one axis might depict acute hypertensive events, another long-term exposure to mild hypertension, and a third BP variability. Clusters of individuals can then be plotted within this space, with proximity indicating similarity in cardiovascular profiles. This allows for rapid identification of high-risk populations or subgroups requiring similar interventions. Visualizing the trajectory of individuals or cohorts within this therapeutic space over time offers unique insights into the dynamics of BP control. For instance, a user's movement toward a low-risk cluster after initiating a new treatment could signify its effectiveness, while stagnation or regression might prompt reevaluation.

Dynamic metrics derived from these visual representations add further value by quantifying changes in the therapeutic space. For example, the velocity of movement within the plot indicates the rate of improvement or deterioration, while acceleration measures how quickly a user responds to an intervention. These metrics can guide the intensity of interventions; for example, a user with slow velocity or minimal acceleration may benefit from a more aggressive treatment approach. Additionally, metrics of distance within the plot can cluster users based on their risk profiles or therapeutic responses. Clustering algorithms, whether automated, semi-automated, or manual, can group individuals with similar BP patterns or responses to interventions, enabling personalized treatment plans.

Clinicians may evaluate the specific risk profiles of users and predict the success of specific treatment options. For example, the risk profile of the user may be used to predict which lifestyle interventions are more likely to be useful (e.g., salt reduction is not useful for certain types risk profiles, but alcohol and weight reduction will be very useful). Likewise, the risk profile of the user may be used to predict which medications will be successful (e.g., an ACE-Inhibitor will be more useful over a calcium channel blocker). In some examples, the risk profile of the user is used to predict the dose of medication required. In some examples, the risk profile of the user is used to predict the likelihood of success of invasive procedures, such as renal denervation.

For individuals undergoing treatment, dynamic trajectory metrics provide a powerful tool for monitoring progress. Clinicians can evaluate how a patient's therapeutic markers evolve in response to an intervention, identifying periods of improvement or stagnation. When applied to cohorts, these metrics enable the comparison of different intervention strategies. In some examples, a group (e.g., clinicians, researchers, and/or insurance companies) can assess overall cohort risk and the success or failure of interventions applied across the group. A/B testing or semi-randomized trials can assess the efficacy of lifestyle modifications, medications, or surgical procedures. By tracking the movement of intervention groups within the therapeutic space, researchers can determine which approaches yield the most significant shifts toward lower-risk regions. Sociodemographic and physiological criteria can also guide the allocation of interventions, ensuring that strategies are tailored to the needs of specific populations.

Visual representations of therapeutic markers can also serve as a foundation for cardiovascular risk profiling. By mapping clusters of users to different risk levels, healthcare providers can prioritize interventions for those at highest risk. For example, users in a cluster associated with frequent acute hypertensive events might receive immediate medication adjustments, while those with chronic mild hypertension could benefit from lifestyle counseling. Risk calculations can be informed by prospective cohort studies or by leveraging existing medical knowledge to associate specific markers with cardiovascular outcomes. The visual plots can then be enhanced with overlays indicating risk zones, where colors or symbols denote varying degrees of cardiovascular risk. Such visual tools not only aid clinicians in decision-making but also improve patient engagement by making complex risk assessments more comprehensible.

Combining therapeutic markers into a single risk score or visual representation offers numerous applications beyond individual care. For public health systems, these tools can identify high-risk populations, allocate resources efficiently, and evaluate the impact of large-scale interventions. For instance, tracking the composite index of a community before and after implementing a hypertension screening program can reveal its effectiveness in reducing overall cardiovascular risk. Similarly, visual plots can identify geographic or demographic disparities in BP control, guiding targeted public health initiatives.

As described above, the integration and combination of advanced therapeutic markers represent a paradigm shift in BP monitoring and hypertension management. Whether through composite indices or visual representations, these tools and resources provide a comprehensive understanding of BP control, enabling tailored interventions and dynamic tracking of therapeutic outcomes. By incorporating time and magnitude into cardiovascular risk assessments, healthcare systems can more effectively address the global burden of hypertension, improving outcomes for individuals and populations alike.

In some examples, the system 100 is configured to calculate Blood Pressure Variability (BPV). BPV is an independent cardiovascular (CV) risk factor often overlooked in traditional hypertension management. Continuous BP monitoring provides an unparalleled opportunity to accurately calculate BPV, offering critical insights into the complex regulation of the cardiovascular system and the interplay of physiological and environmental factors. Unlike sporadic measurements, continuous data allows for precise quantification of BP fluctuations over different time frames, enhancing our understanding of its implications for CV risk and BP control. Poor BPV control is associated with increased end-organ damage, higher rates of stroke, and greater overall cardiovascular morbidity.

To capture BPV comprehensively, the choice of analytical methods depends on the monitoring period and physiological context. Short-term BPV metrics, such as Average Real Variability (ARV), Standard Deviation (SD), and Successive Variation (SV), are effective for evaluating fluctuations over hours to days. Long-term BPV metrics, such as Weighted Standard Deviation (WSD), Variability Independent of the Mean (VIM), and Day-Night BP differences, are better suited for detecting patterns over weeks to months. Day-Night BP differences and spectral analysis can provide insights into the impact of circadian misalignment, which is critical in conditions like shift work or sleep disorders. Integrating BPV as a therapeutic marker through continuous monitoring offers a new dimension in hypertension management. It allows clinicians to identify patients with high BPV, refine risk stratification, and tailor interventions, such as optimizing medication timing, improving sleep hygiene, or implementing stress-reduction strategies. As BPV reflects the dynamic interplay of multiple factors, addressing it directly can improve overall BP control and reduce long-term cardiovascular risk.

In some examples, the system 100 is configured to provide comprehensive BP monitoring for pregnant and postpartum women. Pregnancy and the postpartum period are critical times for BP management, particularly for women at risk of pre-eclampsia or other hypertensive disorders. Continuous BP monitoring offers a transformative approach to addressing these risks by providing detailed, real-time insights into BP patterns throughout pregnancy and beyond. Early detection and management are paramount to improving outcomes for both mother and baby. In some examples, continuous tracking of BP enables early detection of hypertensive patterns, including gestational hypertension or pre-eclampsia, even before symptoms arise. Likewise, monitoring circadian rhythms and nocturnal BP trends helps identify high-risk deviations, often missed with clinic-based or spot-check readings. In some examples, the therapeutic engine 134 provides personalized recommendations, such as lifestyle adjustments, medication timing, and dietary modifications, can be optimized based on continuous data.

With respect to pre-eclampsia risk management, BPV metrics such as ARV and VIM enable the system 100 to provide critical insights into BP fluctuations, a hallmark of pre-eclampsia risk. In some examples, the system 100 provides real-time alerts notifying women and their healthcare providers when BP exceeds safe thresholds, prompting timely interventions. Regarding postpartum care, postpartum hypertension and pre-eclampsia present significant risks to women. The system 100 can provide monitoring for such persistent hypertension. Continuous BP monitoring ensures continuous surveillance during this period when symptoms may resurge or evolve. Night time BP monitoring helps identify residual or new-onset hypertension, a key factor in postpartum complications. In some examples, the system 100 provides integration with other health indicators, such as heart rate variability, sleep quality, and stress levels, offering a holistic view of maternal recovery. Continuous BP monitoring BPM supports the creation of customized care plans that adjust for the unique needs of pregnant and postpartum women, including the timing of medications, activity levels, and follow-up schedules. By leveraging advanced continuous monitoring technologies, the system 100 addresses the dynamic challenges of pregnancy and the postpartum period. This approach empowers women and their healthcare teams with actionable data, enabling early detection, personalized management, and improved maternal and fetal outcomes.

In some examples, the system 100 is configured to provide alarms and/or notifications for sudden BP changes. Sudden changes in BP, or any of therapeutic metrics/markers described above, can be life-threatening and require immediate attention. The system 100 is equipped with real-time alarms and notifications for managing these abrupt fluctuations of these therapeutic markers, providing an early warning system for patients and healthcare providers. In some examples, the system 100 detects when BP exceeds or falls below pre-set thresholds for each of the described therapeutic markers, triggering immediate alerts. For example, a significant BP spike may indicate a hypertensive crisis, while a sharp drop could signal hypotension or shock. In some examples, the alert thresholds are personalized based on the user's baseline BP, health conditions, and risk factors. In some examples, the system 100 tracks the rate at which BP rises or falls, sending alerts if the change occurs too rapidly, even if values remain within the normal range. This helps identify early signs of instability or underlying issues, such as autonomic dysfunction or medication side effects. For extreme changes, such as a sudden systolic BP>180 mmHg or <80 mmHg, the system 100 may automatically escalate notifications to emergency contacts or healthcare providers.

In some examples, by correlating BP changes with contextual data (e.g., physical activity, stress levels, sleep stages), the system 100 can provide insights into possible triggers and help mitigate future episodes. This feature is useful for identifying patterns, such as stress-induced spikes or orthostatic hypotension after standing. In some examples, users and/or healthcare providers can customize the frequency, type (e.g., vibration, sound, or text), and the recipient(s) of alerts to ensure appropriate responses without alarm fatigue. In some examples, after a sudden change, the system 100 provides real-time recommendations (e.g., via the therapeutic engine 134), such as resting, hydrating, or contacting a medical professional, depending on the severity of the fluctuation. By implementing alarms and notifications for sudden BP changes, the system 100 enhances safety, improves early intervention, and empowers users to take timely actions, reducing the risk of severe complications from hypertensive crises, hypotension, or other BP-related events.

### User Engagement

In some embodiments, the system 100 presents physiological information to the user 110 via a graphical interface (e.g., the client application 112) and tracks user engagement with this information. In some examples, the user's engagement is tracked by the engagement engine 126 of the application engine 124. In some examples, the user's engagement is tracked on the first user device 102a (e.g., via the UI engine 113). By monitoring when and how frequently the user 110 interacts with their physiological information, the system 100 can identify individualized engagement patterns or “attention windows.” Utilizing these insights, the system 100 delivers personalized recommendations and interventions tailored to the user's habits and preferences, thereby enhancing adherence to and engagement with treatment plans and promoting healthier behavior.

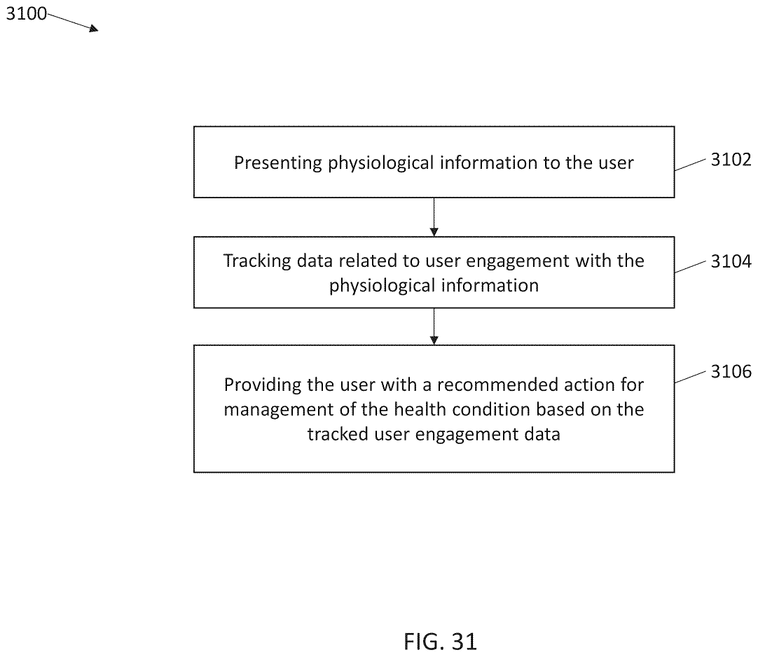

FIG. 31 illustrates a flowchart of a method 3100 for managing a health condition of a user in accordance with aspects described herein. In some examples, the method 3100 is performed, at least in part, by the engagement engine 126.

At step 3102, physiological information is presented to the user 110. In some examples, the client application 112 receives physiological information related to the user's BP condition, such as BP measurements including TTR, HL, and CBPL from the application server 124. This information is presented to the user 110 via a graphical user interface of the client application 112.

At step 3104, the engagement engine 126 tracks data related to the user's engagement with the physiological information. The engagement engine 126 (or UI engine 113) tracks data related to user engagement, including the time of day and frequency of user interactions with the physiological or BP information.

At step 3106, the client application 112 provides the user with a recommended action for management of a health condition based on the tracked user engagement data. By analyzing the engagement data, the engagement engine 126 identifies user-specific attention windows (i.e., times when the user 110 is most likely to engage with their health data). Based on these patterns, the therapeutic engine 134 can provide personalized medication reminders during optimal engagement periods, enhancing adherence to medication schedules. This approach connects the timing of user interactions with their physiological patterns and tailors interventions accordingly.

In some examples, physiological BP information is presented to the user 110 and the engagement engine 126 tracks engagement metrics such as the duration and frequency of interactions. The system 100 analyzes this data alongside the user's lifestyle interventions and BP levels to understand how engagement correlates with health outcomes. By identifying periods when the user 110 is more receptive, the therapeutic engine 134 can deliver tailored lifestyle coaching (e.g., such as exercise suggestions or dietary adjustments) during times when the user 110 is most engaged. This adaptive approach enhances the efficacy of interventions by aligning them with the user's natural engagement patterns.

In some examples, the system 100 uses the tracked engagement data to predict the user's BP responses and potential cardiovascular risks. The engagement engine 126 correlates the timing and frequency of user interactions with fluctuations in BP measurements, including TTR and CBPL. By analyzing these patterns, the system 100 anticipates periods of elevated BP or heightened risk to organs like the kidneys or brain. In some examples, the therapeutic engine 134 can provide proactive recommendations to mitigate adverse outcomes, such as stress management techniques or medical consultations.

In some examples, the engagement engine 126 tracks the user's engagement with physiological BP information and identifies user-specific attention windows. The system 100 may adjust the complexity, tone, and delivery of medical information based on the user's engagement patterns and preferences. For users who engage frequently and for longer durations, the system 100 provides more detailed insights into their BP condition, including advanced metrics like TTR and CBPL. For users with less frequent engagement, the system 100 may offer concise summaries and simplified recommendations. This personalization enhances user understanding and management of their BP condition. In some examples, the UI engine 113 and/or the engagement engine 126 integrates gamification elements into the graphical user interface presenting BP information (e.g., the client application 112). The gamification elements may be personalized based on user-specific data, such as engagement patterns, health metrics, and behavioral trends. This personalization ensures the gamification elements are tailored to enhance user motivation and adherence effectively. By tracking user engagement data such as frequency and duration of interactions, the system 100 may reward users for consistent engagement and achievement of BP targets, like improved TTR and CBPL. In such examples, users may earn badges, points, or progress through levels as they maintain or improve their BP metrics. This approach leverages user behavior tracking to enhance compliance with monitoring and management activities, leading to better BP outcomes.

In some examples, the system 100 uses user engagement data and physiological BP information to predict the user's annual healthcare costs related to their BP condition. By analyzing how engagement patterns correlate with BP control and adherence to management recommendations, the engagement engine 126 estimates potential future costs for the user and payers. This information may be presented to the user 110, emphasizing the financial benefits of sustained engagement and effective BP management. In some examples, the therapeutic engine 134 is configured to recommend actions that include strategies to improve engagement and reduce long-term healthcare expenses.

In some examples, the engagement engine 126 detects signs of engagement fatigue, such as decreased interaction frequency or shorter engagement durations. In response, the engagement engine 126 dynamically adjusts the frequency and/or timing of notifications and recommended actions to prevent overwhelming the user 110. By reducing notification fatigue, the system 100 maintains user engagement over the long term, supporting sustained adherence to BP management strategies. Likewise, the engagement engine 126 tracks user engagement and identifies periods of low interaction with BP information. When decreased engagement is detected, the therapeutic engine 134 may offer social support options, such as connecting the user with caregivers, family members, or online support communities. By providing additional encouragement during times of waning engagement, the system 100 promotes sustained adherence to BP management practices and fosters a supportive environment for the user 110. Similarly, the engagement engine 126 may use predictive analytics to anticipate periods when the user 110 may exhibit low engagement, based on historical patterns of interaction. Before these periods occur, the therapeutic engine 134 proactively delivers motivational content, simplified interventions, or scheduled reminders to encourage continued engagement. This approach reduces barriers to adherence and supports the user's ongoing BP management by addressing potential lapses before they happen.

In some examples, the engagement engine 126 is configured to analyze the timing of user engagement with BP information and correlate it with periods of increased physiological risk, such as elevated BP levels or heart rate. The engagement engine 126 identifies if the user 110 tends to engage during or after periods of high BP and the therapeutic engine 134 provides real-time alerts or interventions to address immediate risks. For instance, if the user 110 frequently checks their BP data during stressful work hours, the therapeutic engine 134 may recommend relaxation techniques or brief exercises to mitigate stress-induced BP spikes. In some examples, the engagement engine 126 is configured to aggregate contextual information, such as the user's age, gender, occupation, exercise habits, and sleep patterns alongside engagement data. In some examples, the contextual information is collected by the user engine 128 and stored in the user data database 136. By understanding the broader context of the user's engagement with BP information, the therapeutic engine 134 can tailor recommended actions to align with their specific circumstances. For example, if a user's engagement drops during business trips, the therapeutic engine 134 may suggest portable stress-relief techniques or provide reminders to check BP despite schedule changes,

In some examples, the engagement engine 126 receives additional physiological information related to other health conditions, such as heart rate, glucose levels, or activity data (e.g., from the external data sources 142, additional devices 144, etc.). By tracking engagement with all health metrics, the engagement engine 126 identifies correlations between user interactions and overall health management. The therapeutic engine 134 may recommend action based on a holistic view of the user's health, enhancing the management of multiple chronic conditions alongside BP. For instance, the therapeutic engine 134 may suggest dietary adjustments that benefit both BP and glucose control.

In some examples, the engagement engine 126 utilizes AI or ML learning algorithms to refine user-specific attention windows based on historical engagement data (e.g., via the AI engine 132). By continuously learning from the user's interaction patterns, including times of day and engagement frequency, the system 100 improves the timing and content of recommended actions. This adaptive approach ensures that interventions are delivered when the user is most receptive, enhancing the effectiveness of BP management. In some examples, the AI model 200 is configured to generate content to be exposed the user 110. Examples of such content include texts, images, videos, or multimodal content to explain the user his diagnosis, to personalize the planned intervention, and to visualize in different manners the evolution of his/her cardiovascular and risk markers. In such examples, the AI model 200 may be, or include, an LLM. The LLM may be combined with reasoning advanced models, or enable real-time unimodal or multimodal conversations, or support several languages. By generating user-specific content, the AI model 200 can improve adherence and engagement of the user 110. In some examples, the AI model 200 is configured to perform further operations or to generate content via several agents to facilitate the healthcare workflows related to the use of the system 100, including but not limited to Appointment Scheduling Agents, Referral Coordination Agents, Prescription Management Agents, Drug Delivery Coordination Agents, Test Scheduling and Tracking Agents, Insurance Authorization Agents, Care Team Communication Agents, Patient Intake Agents, Follow-up and Recall Agents, and Resource Allocation Agents. Any of these agents might be built into the system 100 or accessed from remote systems (e.g., via API calls). The AI model 200 may be pre-trained or connected to data from the system 100, or data from the user 110, or data from a group of users via retrieval-augmented generation (RAG) pipelines.

In some examples, the UI engine 113 presents visualizations via the client application 112 that links the user's engagement patterns with actual physiological outcomes, such as improvements in TTR and CBPL. By showing how consistent engagement and adherence to recommended actions positively impact BP control, the system 100 reinforces beneficial behaviors. This feedback loop encourages users to maintain regular interaction with their BP information and continue following management recommendations.

In some examples, the engagement engine 126 (or UI engine 113) tracks elements of the graphical user interface the user 110 interacts with most frequently, such as trend graphs, educational content, or alerts. By analyzing this data, the UI engine 113 personalizes the interface to highlight preferred features or simplify navigation. For example, if a user often accesses BP trend graphs, the UI engine 113 may display these graphs prominently upon login. This customization enhances user experience, promotes sustained engagement with BP information, and supports effective management of the health condition.

In some examples, the engagement of the user 110 is tracked, and reminders are presented not as graphical or text-based reminders, but as haptic feedback to the user 110. The haptics may come from locations such as the first or second user devices 102a, 102b. In some examples, the engagement of the user 110 is tracked and based on their patterns of behavior, smart pill bottles or dispensers are programmed to be activated based on the user's engagement patterns. This minimizes noncompliance with medication treatments for more successful BP management.

While the above embodiments describe engagement patterns of individual users, it should be appreciated that the engagement engine 126 may track engagement patterns for groups of users. Users may be grouped based on various characteristics such as, for example, demographics, where the users live, time of the year, sociodemographic factors, and the like. In some examples, the groups of users are used to predetermine engagement patterns and/or attention windows for new users. For example, the system 100 may automatically select an engagement pattern for a new user based on engagement patterns from one or more groups of existing users that have similar characteristics as the user. The use of predetermined (or precalculated) engagement patterns may be advantageous when onboarding new users that have limited data (e.g., during the first few days). Once the user has fully onboarded and sufficient data has been collected, the engagement engine 126 may calculate an individualized engagement pattern and/or attention window for the user.

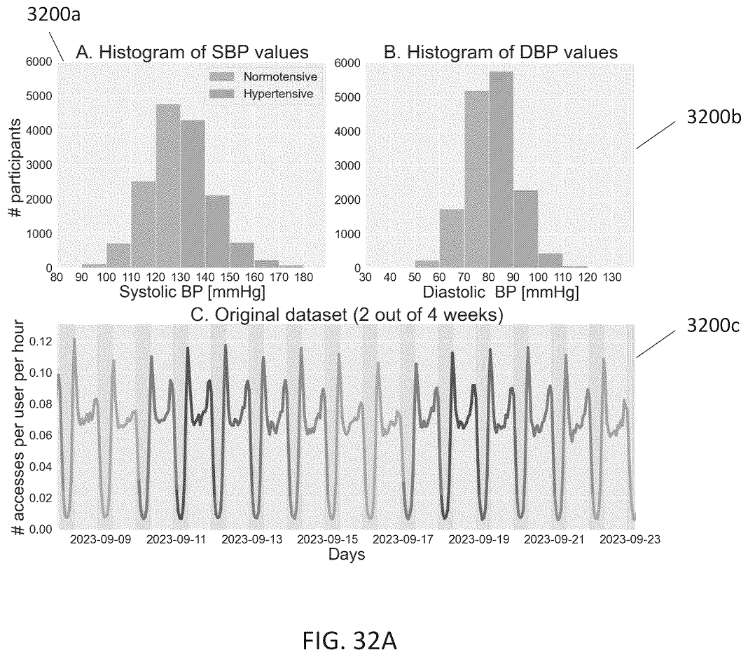

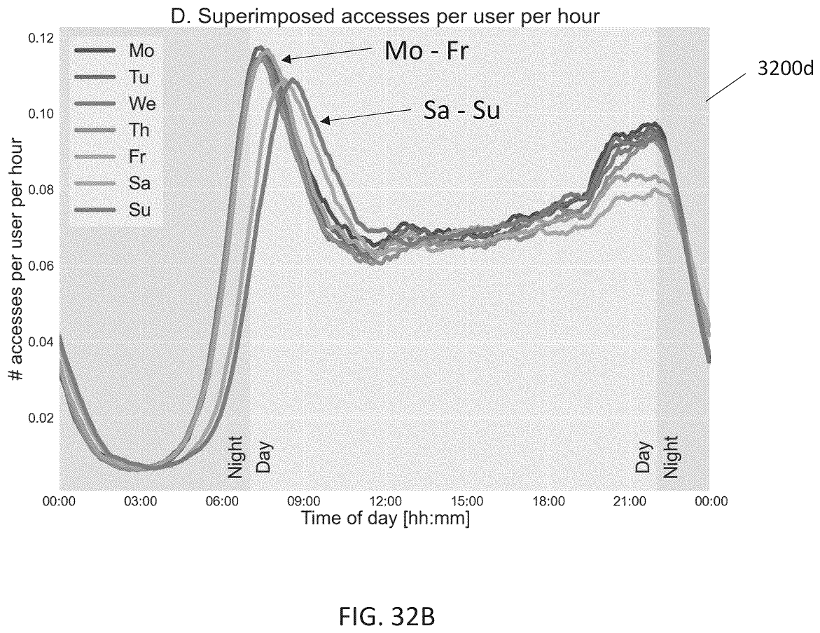

FIG. 32A illustrates several example charts 3200a-3200c corresponding to a group of users. Chart 3200a is a histogram representing the SBP values of the group. Likewise, chart 3200b is a histogram representing the DBP values of the group. In some examples, each histogram includes a single measurement (or value) from each user. As shown, the charts may include color coding to differentiate between normotensive and hypertensive users. In some examples, the engagement engine 126 is configured to retrospectively analyze the number of hourly application accesses by the group of users. In the illustrated example, the engagement engine 126 analyzed application accesses by the group of users for four weeks (e.g., 1-28 Sep. 2023). Chart 3200c is a plot representing the number of hourly synchronizations (or accesses) performed by the group of users over two of the four weeks. In one example, the hourly count was established using a 1-hour centered rolling window with a 5-minute step. Chart 3200c shows clear patterns of behavior of the general group with specific attention windows, one early in the morning and another before bedtime. Similarly, chart 3200d of FIG. 32B illustrates the number of synchronizations performed by hour per user, superimposed by day of the week, highlighting changes in the attention windows according to each day of the week. As shown, a clear weekly pattern of interest from Sunday evening until Friday morning can be seen, with reduced interest in self-monitoring between Friday evening until Sunday morning. There is a noticeable lag of approximately 1 hour on weekend mornings, suggesting users wake up later during weekends.

These results show distinct hourly and weekly patterns of interest that indicate specific attention windows when users typically check their BP values. The engagement patterns suggest the emergence of habitual use, pointing to a potential ‘hooking’ effect with wearable BP monitors. User motivations for uploading data may include checking day/night-time BP variability, checking BP in case of symptoms, generating a report for family members or physicians, long-term monitoring of BP and/or monitoring of changes based on lifestyle changes. Understanding the attention windows when users check their BP enables the development of personalized strategies. For example, gamification may encourage users to engage in activities such as moving, exercising, or taking breaks, leading to improved overall health outcomes and BP reduction. Hourly engagement patterns can also help identify periods when users are likely to engage in behaviors that raise BP, such as consuming high-sodium foods or experiencing stress. For instance, if data suggest an increase in BP for several days, this could be correlated to a decrease in physical activity, a decrease in antihypertensive drug adherence, or a change in diet. Monitoring behavior is often the first step in a change of attitude and reinforces patient empowerment. Furthermore, understanding when users check their BP can aid in developing personalized lifestyle interventions. For example, if a user checks their BP after a workout, the system 100 may provide positive reinforcement, such as congratulatory messages or tips on maintaining the workout routine. Conversely, if a user's BP readings remain high despite regular checks, the system 100 might suggest increasing physical activity, making dietary adjustments, or consulting a healthcare provider. These strategies could positively influence user habits, paving the way for the creation of targeted, data-driven interventions that can substantially improve hypertension management.

In some examples, the configuration of the system 100 is adaptable based on the geographic location of user 110. For example, the configuration of the system 100 may vary based on local regulations, regulatory approval processes, or regulatory compliance requirements. Since BP is a medical parameter, some features of the system 100 may require regulatory approval before being activated. However, the user engagement, adherence, and lifestyle modification features described above do not necessarily need to be approved by regulatory authorities. As such, the system 100 may be configured initially to provide features that do not require regulatory approval. In such examples, the system 100 may be initially configured to monitor engagement and provide recommendations accordingly. As approvals are received for other features (e.g., BP monitoring), the configuration of the system 100 is adapted to unlock these features for users (e.g., via an update to the client application 112). This adaptable configuration is advantageous because it allows the system 100 to easily evolve without needing to notify to a regulatory body every time a change or update is being performed to an unregulated feature, facilitating the deployment of the system 100 to meet diverse regulatory compliance requirements. Various components of the system 100 may be specifically configured to adhere to distinct local regulatory standards, including obtaining medical certification approvals for hardware and software. This includes compliance with frameworks and approvals associated with software classified as a medical device (SaMD) or medical device software.

As described above, the system 100 provides for early and widespread detection of BP-related health issues. User-initiated screenings increase accessibility and encourage proactive health monitoring, enabling early identification of at-risk individuals. Automated, passive, and continuous monitoring provides a detailed BP profile over time, improving diagnostic accuracy and allowing for the use of advanced therapeutic markers that assess both the time and magnitude of BP deviations from target ranges. The system 100 tracks user engagement to facilitate the delivery of tailored interventions during optimal times, enhancing user adherence and the efficacy of the management plan. The system 100 may classify users according to personalized risk profiles and can adjust management strategies based on individual goals, such as minimizing the risk of stroke in older adults or reducing long-term disease risks in younger populations. By integrating game-like elements such as progress tracking, rewards, or challenges tied to BP values, advanced therapeutic markers, and/or user engagement data, users are motivated to take proactive action. This approach encourages lifestyle changes (e.g., reducing salt intake, increasing physical activity, etc.) by making the process engaging and rewarding, helping users actively target healthier BP levels. In some examples, the system 100 utilizes an LLM to create personalized multimedia content. This approach transforms rewards, challenges, and gamification elements into engaging, customized materials that resonate with the user. By generating dynamic text, videos, images, and sounds tailored to the user's preferences and progress, the system 100 provides a canonical overview of the user's health journey over time. This personalized multimedia experience enhances motivation, sustains engagement, and promotes adherence to hypertension management plans.

### Hardware and Software Implementations

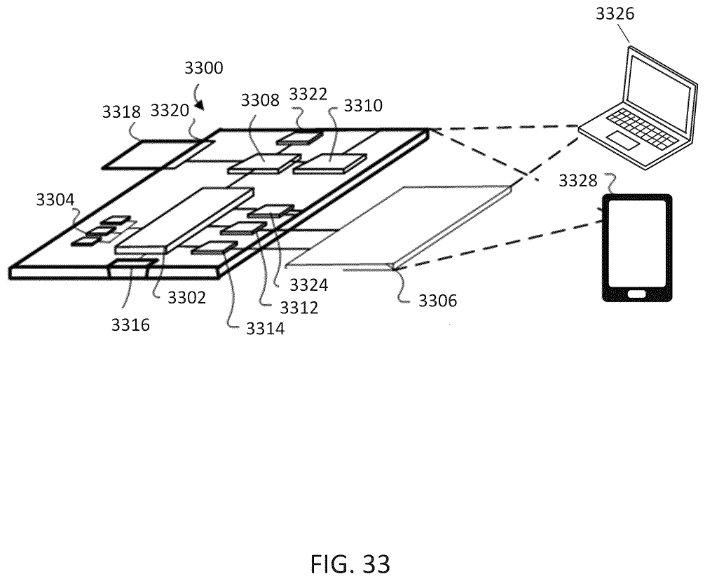

FIG. 33 shows an example of a generic computing device 3300, which may be used with some of the techniques described in this disclosure (e.g., as user devices 102a, 102b or application server 124). Computing device 3300 includes a processor 3302, memory 3304, an input/output device such as a display 3306, a communication interface 3308, and a transceiver 3310, among other components. The device 3300 may also be provided with a storage device, such as a micro-drive or other device, to provide additional storage. Each of the components 3300, 3302, 3304, 3306, 3308, and 3310, are interconnected using various buses, and several of the components may be mounted on a common motherboard or in other manners as appropriate.

The processor 3302 can execute instructions within the computing device 3300, including instructions stored in the memory 3304. The processor 3302 may be implemented as a chipset of chips that include separate and multiple analog and digital processors. The processor 3302 may provide, for example, for coordination of the other components of the device 3300, such as control of user interfaces, applications run by device 3300, and wireless communication by device 3300.

Processor 3302 may communicate with a user through control interface 3312 and display interface 3314 coupled to a display 3306. The display 3306 may be, for example, a TFT LCD (Thin-Film-Transistor Liquid Crystal Display) or an OLED (Organic Light Emitting Diode) display, or other appropriate display technology. The display interface 3314 may comprise appropriate circuitry for driving the display 3306 to present graphical and other information to a user. The control interface 3312 may receive commands from a user and convert them for submission to the processor 3302. In addition, an external interface 3316 may be provided in communication with processor 3302, so as to enable near area communication of device 3300 with other devices. External interface 3316 may provide, for example, for wired communication in some implementations, or for wireless communication in other implementations, and multiple interfaces may also be used.

The memory 3304 stores information within the computing device 3300. The memory 3304 can be implemented as one or more of a computer-readable medium or media, a volatile memory unit or units, or a non-volatile memory unit or units. Expansion memory 3318 may also be provided and connected to device 3300 through expansion interface 3320, which may include, for example, a SIMM (Single In Line Memory Module) card interface. Such expansion memory 3318 may provide extra storage space for device 3300, or may also store applications or other information for device 3300. Specifically, expansion memory 3318 may include instructions to carry out or supplement the processes described above, and may include secure information also. Thus, for example, expansion memory 3318 may be provided as a security module for device 3300, and may be programmed with instructions that permit secure use of device 3300. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 3304, expansion memory 3318, memory on processor 3302, or a propagated signal that may be received, for example, over transceiver 3310 or external interface 3316.

Device 3300 may communicate wirelessly through communication interface 3308, which may include digital signal processing circuitry where necessary. Communication interface 3308 may in some cases be a cellular modem. Communication interface 3308 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 3310. In addition, short-range communication may occur, such as using a Bluetooth, WiFi, or other such transceiver (not shown). In addition, GPS (Global Positioning System) receiver module 3322 may provide additional navigation- and location-related wireless data to device 3300, which may be used as appropriate by applications running on device 3300.

Device 3300 may also communicate audibly using audio codec 3324, which may receive spoken information from a user and convert it to usable digital information. Audio codec 3324 may likewise generate audible sound for a user, such as through a speaker, e.g., in a handset of device 3300. Such sound may include sound from voice telephone calls, may include recorded sound (e.g., voice messages, music files, etc.) and may also include sound generated by applications operating on device 3300. In some examples, the device 3300 includes a microphone to collect audio (e.g., speech) from a user. Likewise, the device 3300 may include an input to receive a connection from an external microphone.

The computing device 3300 may be implemented in a number of different forms, as shown in FIG. 33. For example, it may be implemented as a computer (e.g., laptop) 3326. It may also be implemented as part of a smartphone 3328, smart watch, tablet, personal digital assistant, or other similar mobile device.

Some implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation.

Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1-18. (canceled)

19. A method for managing a health condition of a user comprising:
receiving physiological information related to the health condition of the user;
presenting the physiological information to the user;
tracking data related to user engagement with the physiological information; and
providing the user with a recommended action for management of the health condition based on the tracked user engagement data.

20. The method of claim 19, wherein the health condition comprises a BP condition.

21. The method of claim 19, wherein the physiological information comprises a BP measurement.

22. The method of claim 21, wherein the BP measurement comprises at least one of TTR and CBPL.

23. The method of claim 19, wherein presenting the physiological information to the user comprises presenting the information on a graphical user interface.

24. The method of claim 19, wherein the user engagement data comprises a time when the user engages with the physiological data.

25. The method of claim 24, wherein the time comprises at least one of a time of day, a day of the week, a week of the month, and a day of the year.

26. The method of claim 19, wherein the user engagement data further comprises at least one of frequency and length of engagement for when the user engages with the physiological information.

27. The method of claim 19, wherein the user engagement data further comprises an activity that the user performs when the user is engaging with the physiological information.

28. The method of claim 19, wherein providing the user with the recommended action for management of the health condition based on the tracked user engagement data comprises determining the recommended action based on an engagement pattern of the user.

29. The method of claim 28, wherein the engagement pattern of the user is determined based on the received physiological information related to the health condition of the user.

30. The method of claim 28, wherein the engagement pattern of the user is determined based on an engagement pattern or physiological information of at least one other user.
